(19) United States
(12) Patent Application Publication
(10) Pub. No.: US 2025/0377354 A1
(43) Pub. Date: Dec. 11, 2025

(54) ANALYTE CHARACTERIZATION BY DIFFERENTIAL BINDING OF BINDING REAGENTS

(71) Applicant: Nautilus Subsidiary, Inc., Seattle, WA (US)

(72) Inventors: Zhengjian ZHANG, San Carlos, CA (US); Gajalakshmi DAKSHINAMOORTHY, San Jose, CA (US); Parag MALLICK, San Carlos, CA (US); Sanjib GUHA, Foster City, CA (US); Maryam JOUZI, San Francisco, CA (US)

(21) Appl. No.: 19/226,698

(22) Filed: Jun. 3, 2025

Related U.S. Application Data
(60) Provisional application No. 63/656,795, filed on Jun. 6, 2024.

(51) Int. Cl. G01N 33/53; G01N 33/68

(52) U.S. Cl. CPC G01N 33/5304; G01N 33/68

(57) ABSTRACT

Methods of characterizing analytes such as protein molecules by differential association or dissociation of binding reagents are provided. Observation of association or dissociation of binding reagents from analytes under two or more differing association or dissociation conditions can facilitate characterization of the analytes due to observed differences in binding patterns.

### RELATED U.S. APPLICATIONS

This application claims priority to U.S. Provisional Application No. 63/656,795, filed on Jun. 6, 2024, which is incorporated herein by reference in its entirety.

### BACKGROUND

A binding reagent can include any molecule or particle that can form a binding interaction with a target molecule. Binding reagents can include affinity agents (e.g., antibodies, antibody fragments, aptamers, mini-peptide binders, etc.) as well as other molecules or particles that can form a covalent or non-covalent binding interaction with a target molecule. The characteristics of a binding interaction between a binding reagent and a target molecule can depend upon the chemical nature of the binding reagent, the chemical nature of the target molecule, and/or the chemical environment that mediates the binding interaction.

Target molecules may be characterized by the observation of binding interactions between the target molecules and binding reagents. In some cases, binding interactions between target molecules and binding reagents may be directly observable via the observation of complexes formed between a target molecule and a binding reagent. In other cases, binding interactions may be observable by a secondary observation of a target molecule, such as observing a chemical modification or morphological change caused by a binding interaction between the target molecule and a binding reagent.

Observation of binding interactions between target molecules and binding reagents may be useful for characterizing target molecules. In some cases, observation of binding interactions between binding reagents and unknown molecules may provide information on the chemical nature of the unknown molecules, thereby providing some amount of characterization or identification of the unknown molecules. In other cases, observation of binding interactions between binding reagents and known molecules may provide information on previously unknown or unobserved interactions of either the molecules or binding reagents.

### SUMMARY

In an aspect, provided herein is a method, comprising: a) detecting in a first fluidic medium the presence or absence of binding of binding reagents of a first plurality of binding reagents to a plurality of molecules, wherein the first fluidic medium has a first fluidic condition, b) detecting in a second fluidic medium the presence or absence of binding of the binding reagents of a second plurality of binding reagents to the plurality of molecules, wherein the second fluidic medium has a second fluidic condition, wherein the first fluidic condition of the first fluidic medium differs from the second fluidic condition of the second fluidic medium, and c) characterizing an individual molecule of the plurality of molecules based upon a binding pattern of binding reagents of the plurality of binding reagents to the individual molecule of the plurality of molecules, wherein the binding pattern comprises a presence or absence of binding of a binding reagent of the plurality of binding reagents to the individual molecule for the first fluidic condition and the second fluidic condition.

In another aspect, provided herein is a method, comprising: a) detecting in a first fluidic medium the presence or absence of binding of binding reagents of a first plurality of binding reagents to a plurality of molecules, wherein the first fluidic medium has a first fluidic condition, b) detecting in a second fluidic medium the presence or absence of binding of the binding reagents of a second plurality of binding reagents to the plurality of molecules, wherein the second fluidic medium has a second fluidic condition, wherein the first fluidic condition differs from the second fluidic condition, and c) distinguishing a first molecule of the plurality of molecules from a second molecule of the plurality of molecules based upon a binding pattern of the binding reagents of the plurality of binding reagents to the plurality of molecules, wherein the binding pattern comprises for each individual molecule of the plurality of molecules a presence or absence of binding of a binding reagent of the plurality of binding reagents to the individual molecule for the first fluidic condition and the second fluidic condition.

In another aspect, provided herein is a system for analyte characterization, comprising: (a) a fluidic vessel comprising a plurality of molecules, (b) a binding reagent kit comprising a plurality of vessels, wherein each vessel of the plurality of vessels comprises a fluidic medium comprising a plurality of binding reagents, (c) a fluidic system that is configured to transfer the fluidic medium comprising the plurality of binding reagents to the fluidic vessel comprising the plurality of molecules, and (d) a detection system that is configured to detect at single-analyte resolution for each individual molecule of the plurality of molecules a presence or absence of binding of a binding reagent of the plurality of binding reagents.

In another aspect, provided herein is a kit comprising a plurality of vessels, wherein each vessel of the plurality of vessels comprises a fluidic medium, wherein the fluidic medium comprises a plurality of binding reagents, and wherein the kit is further characterized by at least one of: (a) a vessel comprising a first plurality of binding reagents in a first fluidic medium and a different vessel comprising a second plurality of binding reagents in a second fluidic medium, wherein the first plurality of binding reagents is identical to the second plurality of binding reagents, and wherein the first fluidic medium differs from the second fluidic medium, (b) a vessel comprising a first plurality of binding reagents in a first fluidic medium and a different vessel comprising a second plurality of binding reagents in a second fluidic medium, wherein a concentration of the first plurality of binding reagents in the first fluidic medium differs from a concentration of the second plurality of binding reagents in the second fluidic medium, and wherein the first fluidic medium is identical to the second fluidic medium, (c) a vessel comprising a first plurality of binding reagents in a first fluidic medium and a different vessel comprising a second plurality of binding reagents in a second fluidic medium, wherein the first plurality of binding reagents differs from the second plurality of binding reagents, and wherein the first fluidic medium differs from the second fluidic medium, and (d) a vessel comprising a first plurality of binding reagents in a first fluidic medium and a different vessel comprising a second fluidic medium, wherein the second fluidic medium is substantially devoid of binding reagents.

In another aspect, provided herein is a kit comprising at least 20 vessels, wherein each vessel of the at least 20 vessels comprises a fluidic medium, wherein the fluidic medium comprises a plurality of binding reagents, and wherein the kit is further characterized by at least one of: (a) a vessel of the at least 20 vessels comprising a first plurality of binding reagents in a first fluidic medium and a different vessel of the at least 20 vessels comprising a second plurality of binding reagents in a second fluidic medium, wherein the first plurality of binding reagents is identical to the second plurality of binding reagents, and wherein the first fluidic medium differs from the second fluidic medium, (b) a vessel of the at least 20 vessels comprising a first plurality of binding reagents in a first fluidic medium and a different vessel of the at least 20 vessels comprising a second plurality of binding reagents in a second fluidic medium, wherein a concentration of the first plurality of binding reagents in the first fluidic medium differs from a concentration of the second plurality of binding reagents in the second fluidic medium, and wherein the first fluidic medium is identical to the second fluidic medium, and (c) at least 10 vessels containing 10 differing pluralities of binding reagents, wherein each of the 10 differing pluralities of binding reagents differs from each other plurality of binding reagents of the at least 10 differing pluralities of binding reagents with respect to binding reagent structure, binding specificity, or concentration.

### INCORPORATION BY REFERENCE

All publications, items of information available on the internet, patents, and patent applications cited in this specification are herein incorporated by reference to the same extent as if each individual publication, patent, or patent application were specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIG. 1A depicts an association of binding reagents to molecules under two differing binding reagent association conditions, in accordance with some embodiments. FIG. 1B depicts dissociation of binding reagents from molecules under two differing binding reagent dissociation conditions, in accordance with some embodiments.

FIG. 2A displays an association of binding reagents to molecules under two differing binding reagent association conditions when the association conditions are provided sequentially, in accordance with some embodiments. FIG. 2B displays dissociation of binding reagents from molecules under two differing binding reagent dissociation conditions when the dissociation conditions are provided sequentially, in accordance with some embodiments.

FIG. 3 shows a flow chart for a method of characterizing one or more molecules with two or more binding reagent association conditions, in accordance with some embodiments.

FIG. 4 presents a flow chart for a method of characterizing one or more molecules with two or more binding reagent dissociation conditions, in accordance with some embodiments.

FIGS. 5A, 5B, and 5C depict steps of a method of characterizing one or more molecules using multiplexed binding reagents, in accordance with some embodiments.

FIGS. 6A, 6B, 6C, and 6D display steps of a method of transferring a detectable label from a binding reagent to a molecule, in accordance with some embodiments.

FIG. 7A shows dissociation data for a binding reagent dissociating from phosphorylated protein molecules in the presence of a binding reagent dissociation medium containing CHAPS surfactant. FIG. 7B shows dissociation data for a binding reagent dissociating from phosphorylated protein molecules in the presence of a binding reagent dissociation medium containing sodium dodecyl sulfate, sodium acetate, and magnesium chloride at pH 4.25.

FIGS. 8A and 8B present data for dissociation of binding reagents from differing trimer amino acid targets utilizing dissociation mediums containing CHAPS and guanidinium hydrochloride, respectively.

FIG. 9 illustrates a system that is configured to perform certain methods of analyte characterization set forth herein, in accordance with some embodiments.

### DETAILED DESCRIPTION

Binding reagents can include molecules or particles that are delivered during an assay or method and form binding interactions with other molecules or particles as at least part of their activity or function during the assay or method. In some cases, a binding interaction between a binding reagent and a molecule may be directly observable if the binding reagent comprises a detectable label (e.g., a fluorophore, luminophore, radiolabel, etc.). For example, binding of affinity-based binding reagents (e.g., antibodies, aptamers, mini-peptide binders, etc.) to proteins or other analytes may be directly observable if the binding reagents produce fluorescent signals. In other cases, a binding interaction between a binding reagent and a molecule may be indirectly observable, for example by observation of a modification to the molecule caused by the binding reagent. For example, modifications to proteins or other analytes caused by enzymatic binding reagents may be observable after the enzyme has bound to the protein or analyte.

The formation of a complex between a molecule and a binding reagent can be a complex interaction that is affected by the chemical properties and/or structures of the respective molecule and binding reagent, as well as the surrounding chemical environment that mediates the interaction. For a fixed pair of a molecule and a binding reagent that are known to form a complex, the affinity or avidity of the interaction between the molecule and the binding reagent may be modulated by the surrounding chemical environment. For example, changes in pH, ionic strength, temperature, and/or fluid velocity can alter the strength of the interaction between a molecule and a binding reagent. Depending upon the type of detection instrument used to observe complex formation between the molecule and the binding reagent, in some cases, complex formation between a molecule and a binding reagent may be too weak to be observed during the time scale of detection in a particular chemical environment. Conversely, in some cases, complex formation between a molecule and a binding reagent may be sufficiently strengthened to be observed during the time scale of detection in a particular chemical environment.

Accordingly, modulation of binding interactions via alteration of chemical environment may be a useful method for characterizing molecules, binding reagents, or both. For example, a characteristic of a first molecule and/or a second molecule may be differentiated by observing binding interactions or lack thereof between binding reagents and the first and second molecule in the presence of differing chemical environments. The binding reagent may be observed to bind or not bind to both of the first molecule and the second molecule in the presence of a first chemical environment, and bind to only one of the first molecule and the second molecule in the presence of the second chemical environment, thereby differentiating the two molecules. In another example, a binding reagent may be characterized by observing interactions between molecules and a plurality of the binding reagent in the presence of two or more differing chemical environments, thereby determining a set of molecules to which the binding reagent associates for each of the two or more chemical environments.

A binding reagent may be capable of individually forming a binding interaction with two or more differing molecules, but the nature of the binding interaction formed by the binding reagent with each individual molecule of the two or more differing molecules may differ, for example with respect to the on-rate of binding to the individual molecules, the off-rate of binding to the individual molecules, the reactivity of individual molecules when coupled to the binding reagent, or the rate of reaction of individual molecules when coupled to the binding reagent. Accordingly, observation of differences in binding between binding reagents and two or more differing molecules in the presence of two or more fluidic conditions may facilitate differential characterization of the two or more differing molecules. For example, a binding reagent may be observed to dissociate from both of two differing molecules in the presence of a fluid having a first pH but may be observed to only dissociate from one of the two differing molecules in the presence of a fluid having a differing second pH. Observing the binding interactions of both differing molecules under both fluidic conditions facilitates characterization of the differing chemical properties of the molecules due to the differences in binding behavior.

Provided herein are methods and systems for utilizing modulation of binding reagent association or dissociation conditions to characterize molecules. Some methods may utilize identifying changes in a binding interaction between a molecule and a binding reagent under two differing fluidic conditions. Other methods may comprise identifying changes in binding interactions between molecules and binding reagents when the binding reagents include sets of divergent binding reagents.

### Definitions

Terms used herein will be understood to take on their ordinary meaning in the relevant art unless specified otherwise. Several terms used herein and their meanings are set forth below.

In some embodiments set forth herein, the term “address” can refer to a location in an array where a particular molecule (e.g. protein, peptide or unique identifier label) is present. An address can contain a single molecule, or it can contain a population of several molecules of the same species (i.e. an ensemble of the analytes). Alternatively, an address can include a population of different molecules. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses

In some embodiments set forth herein, the term “affinity agent” can refer to a molecule or other substance that is capable of specifically or reproducibly binding to a molecule (e.g. protein) without permanently altering the molecule after the affinity agent dissociates. An affinity reagent can be larger than, smaller than, or the same size as the molecule. An affinity reagent may form a reversible or irreversible bond with a molecule. An affinity reagent may bind with a molecule in a covalent or non-covalent manner. Affinity reagents may include reactive affinity reagents or non-reactive affinity reagents (e.g., antibodies or fragments thereof). Affinity reagents that can be particularly useful for binding to proteins include, but are not limited to, antibodies or functional fragments thereof (e.g., Fab′ fragments, F(ab′)2 fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof.

In some embodiments set forth herein, the term “anchoring moiety” or “anchoring group” can refer synonymously to a molecule or particle that serves as an intermediary attaching a molecule to a surface (e.g., on a solid support or a microbead). An anchoring group may be covalently or non-covalently attached to a surface and/or a polypeptide. An anchoring group may be a biomolecule, polymer, particle, nanoparticle, or any other entity that is capable of attaching to a surface or molecule. An anchoring moiety may provide physical separation between a surface of a solid support and a molecule that is attached to the anchoring moiety. An anchoring moiety may comprise a linking moiety (e.g., a rigid linker, a flexible linker) that attaches a molecule to the anchoring moiety, and optionally provides physical separation between the molecule and a surface of a solid support. An anchoring moiety may comprise a particle or a bead. In some cases, an anchoring group may be a nucleic acid nanoparticle such as a SNAP (e.g., a nucleic acid origami, a nucleic acid nanoball). In some cases, an anchoring group may comprise a non-nucleic acid nanoparticle, such as a polymer nanoparticle, a branched polymer nanoparticle, or a dendrimeric nanoparticle.

In some embodiments set forth herein, the terms “molecule” and “analyte” can refer synonymously to a discrete entity that can be characterized by an interaction with a binding reagent. A molecule may comprise a plurality of atoms joined by covalent bonding. A molecule can include one or more non-covalent bonds that form two-dimensional or three-dimensional structures. A molecule may include a nanoparticle or particle. A molecule may comprise a plurality of polymerized residues. A molecule may be considered a small molecule if the molecule has a molecular weight of less than 1 kiloDalton (kDa). A molecule may be considered a macromolecule if the molecule has a molecular weight of at least 1 kDa.

In some embodiments set forth herein, the term “array” can refer to a population of molecules or analytes (e.g. proteins) that are associated with unique identifiers such that the molecules can be distinguished from each other. A unique identifier can be, for example, a solid support (e.g. particle or bead), address on a solid support, tag, label (e.g. luminophore), or barcode (e.g. nucleic acid barcode) that is associated with a molecule and that is distinct from other identifiers in the array. Molecules can be associated with unique identifiers by attachment, for example, via covalent bonds or non-covalent bonds (e.g. ionic bond, hydrogen bond, van der Waals forces, electrostatics etc.). An array can include different molecules that are each attached to different unique identifiers. An array can include different unique identifiers that are attached to the same or similar molecules. An array can include separate solid supports or separate addresses that each bear a different analyte, wherein the different molecules can be identified according to the locations of the solid supports or addresses.

In some embodiments set forth herein, the term “attached” can refer to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

In some embodiments set forth herein, the terms “binding profile” and “binding pattern” can refer synonymously to a plurality of binding or dissociation outcomes for a molecule with one or more binding reagents. The binding outcomes can be obtained from independent binding or dissociation observations, for example, independent binding outcomes can be acquired using different binding reagents, respectively. Alternatively, the binding outcomes can be generated in silico, for example, being derived from a modification of an empirically obtained binding outcome. A binding profile can include empirical measurement outcomes, candidate measurement outcomes, calculated measurement outcomes, theoretical measurement outcomes or a combination thereof. A binding profile can exclude one or more of empirical measurement outcomes, candidate measurement outcomes, calculated measurement outcomes, or theoretical measurement outcomes or putative measurement outcomes. A binding profile can include a vector of binding outcomes.

In some embodiments set forth herein, the term “binding reagent” can refer to a molecule, particle, or moiety that can form a binding interaction with another molecule as at least part of its activity or function. A binding reagent may form a covalent interaction or a non-covalent interaction with a molecule. A binding reagent may covalently modify a molecule to which the binding reagent binds. A binding reagent may comprise an affinity agent, as set forth herein. A binding reagent may comprise two or more affinity agents. A binding reagent may comprise two or more detectable labels. A binding reagent may comprise a particle (e.g., a nucleic acid nanoparticle, a polymer nanoparticle, a branched or dendrimeric nanoparticle) that couples one or more affinity agents to one or more detectable labels. Examples of multivalent binding reagents (i.e., binding reagents comprising two or more moieties configured to form binding interactions) are described in U.S. Pat. No. 11,692,217 and U.S. patent application No. 20230090454, each of which is herein incorporated by reference in its entirety.

In some embodiments set forth herein, the term “binding specificity” can refer to the tendency of a binding reagent to preferentially bind to a given molecule or molecules relative to other molecules. A binding reagent may have a calculated, observed, known, or predicted binding specificity for a given molecule. Binding specificity may refer to selectivity for a single molecule in a given sample relative to one, some or all other molecules in the sample. Moreover, binding specificity may refer to selectivity for a subset of molecules in a given sample relative to at least one other molecule in the sample.

The term “comprising” is intended herein to be open-ended, including not only the recited elements, but further encompassing any additional elements.

In some embodiments set forth herein, the term “dissociation specificity” can refer to the tendency of a binding reagent to preferentially dissociate from a given molecule or molecules relative to other molecules. A binding reagent may have a calculated, observed, known, or predicted dissociation specificity for a given molecule. Dissociation specificity may refer to selectivity for a single molecule in a given sample relative to one, some or all other molecules in the sample. Moreover, dissociation specificity may refer to selectivity for a subset of molecules in a given sample relative to at least one other molecule in the sample.

In some embodiments set forth herein, the term “divergent,” when used in reference to binding specificity or dissociation specificity, can refer to a first binding reagent and a second binding reagent having an observable difference in binding specificity or dissociation specificity with respect to a plurality of molecules. A first binding reagent may have a divergent binding specificity or dissociation specificity from that of a second binding reagent if the first binding reagent binds or dissociates from one or more molecules of a plurality of molecules that the second binding reagent does not bind to or dissociate from. A first binding reagent may have a divergent binding specificity or dissociation specificity from that of a second binding reagent if the first binding reagent binds to or dissociates from an epitope, moiety, or other structural element present in one or more molecules of a plurality of molecules that the second binding reagent does not bind to or dissociate from. A first binding reagent may have a divergent binding specificity or dissociation specificity from that of a second binding reagent if the first binding reagent has a higher probability of binding to or dissociating from one or more molecules of a plurality of molecules relative to the probability that the second binding reagent binds to or dissociates from the one or more molecules.

In some embodiments set forth herein, the term “each,” when used in reference to a collection of items, is intended to identify an individual item in the collection but does not necessarily refer to every item in the collection. Exceptions can occur if explicit disclosure or context clearly dictates otherwise.

In some embodiments set forth herein, the term “epitope” can refer to a binding target within a protein, polypeptide, or other molecule. Epitopes of polypeptides may include amino acid sequences that are sequentially adjacent in the primary structure of a protein. Epitopes of polypeptides may include amino acids that are structurally adjacent in the secondary, tertiary or quaternary structure of a protein despite being non-adjacent in the primary sequence of the protein. An epitope can be, or can include, a moiety of protein that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other binding reagent. An epitope can optionally bind an antibody to elicit an immune response. However, an epitope need not necessarily participate in, nor be capable of, eliciting an immune response.

In some embodiments set forth herein, the term “fluidic condition” can refer to the chemical and/or physical properties of a fluidic medium comprising a plurality of binding reagents. Fluidic condition of a fluidic medium can be described by one or more of: i) chemical composition of the fluid; ii) weight percentage, volume percentage, molarity, or concentration of fluid chemical components; iii) fluid pH; iv) fluid ionic strength; v) fluid temperature; vi) fluid pressure; vii) fluid velocity; viii) fluid magnetic field orientation and/or magnitude; ix) fluid electrical field orientation and/or magnitude; x) fluid depth or film thickness; xi) binding reagent concentration; and xii) combinations thereof. Chemical composition can include the chemical components of a fluidic medium excluding the binding reagents, including solvents (e.g., aqueous solvents, non-aqueous solvents), co-solvents, ionic compounds, buffering species, surfactant species, chaotropic species, acidifying agents, alkalizing agents, blocking agents (e.g., bovine serum albumins, polymeric blocking reagents, etc.), kosmotropic species, and excipient reagents (e.g., anti-flocculants, anti-freeze agents, antibacterial compounds, etc.). A first fluidic condition may be considered to differ from a second fluidic condition if the first fluidic condition and the second fluidic condition differ with respect to at least one of the foregoing fluid properties.

In some embodiments set forth herein, the terms “group” and “moiety” may be synonymous when used in reference to the structure of a molecule. The terms can refer to a component or part of the molecule. The terms do not necessarily denote the relative size of the component or part compared to the rest of the molecule, unless indicated otherwise.

In some embodiments set forth herein, the term “immobilized,” when used in reference to a molecule that is in contact with a fluid phase, refers to at least part of the molecule being prevented from diffusing in the fluid phase. For example, immobilization can occur due to the molecule being confined at, or attached to, a solid phase. Immobilization can be temporary (e.g. for the duration of one or more steps of a method set forth herein) or permanent. Immobilization can be reversible or irreversible under conditions utilized for a method, system or composition set forth herein.

In some embodiments set forth herein, the term “label” can refer to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

In some embodiments set forth herein, the term “nucleic acid origami” can refer to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

In some embodiments set forth herein, the term “nucleic acid tag” can refer to a nucleic acid molecule or sequence that is encoded with information that uniquely identifies an object with which it is associated. A nucleic acid tag can be associated with an object via a connection. The connection can be physical, including, for example, attachment, colocalization, diffusional contact or the like. Non-physical connections can include, for example, knowledge of a past interaction, knowledge of a shared characteristic, knowledge of common manipulations, knowledge of origin or the like. The nucleic acid tag can be, for example, DNA, RNA or analogs thereof. The length of the tag sequence can be at least about 5, 8, 10, 15, 20, 25, 30, 40, 50, 75, 100 or more nucleotides. Alternatively or additionally, the length of the tag sequence can be at most about 100, 75, 50, 40, 30, 25, 20, 15, 10, 8, 5 or fewer nucleotides.

In some embodiments set forth herein, the term “post-translational modification” can refer to a change to the chemical composition of a protein compared to the chemical composition encoded by the gene for the protein. Exemplary changes include those that alter the presence, absence or relative arrangement of different regions of amino acid sequence (e.g., splicing variants, or protein processing variants of a single gene), or due to the presence or absence of different moieties on particular amino acids (e.g., post-translationally modified variants of a single gene). A post-translational modification can be derived from an in vivo process or in vitro process. A post-translational modification can be derived from a natural process or a synthetic process. Exemplary post-translational modifications include those classified by the PSI-MOD ontology. See Smith, L. M. et al. Nat. Methods, 2013, 10, 186-187.

In some embodiments set forth herein, the terms “protein” and “polypeptide” can refer synonymously to a molecule comprising two or more amino acids joined by a peptide bond. A protein may also be referred to as a polypeptide, oligopeptide or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

In some embodiments set forth herein, the term “single,” when used in reference to an object such as an molecule or analyte, can mean that the object is individually manipulated or distinguished from other objects. A single analyte can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

In some embodiments set forth herein, the term “solid support” can refer to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

In some embodiments set forth herein, the term “structured nucleic acid particle” or “SNAP” can refer to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

In some embodiments set forth herein, the term “type,” when used in reference to a subset of molecules, can refer to a characteristic that is shared by the molecules in the subset and that distinguishes the molecules in the subset from molecules that are not in the subset. The characteristic can be any of a variety of characteristics known for the molecules. Any of a variety of molecules can be categorized by type, including, for example, proteins. Exemplary characteristics that can be used to categorize proteins by type include, but are not limited to, amino acid composition, full length amino acid sequence, proteoform, presence or absence of an amino acid sequence motif, number of amino acids present (i.e. sequence length), molecular weight, presence or absence of a particular epitope, presence or absence of epitope(s) recognized by a particular affinity reagent, probability of binding a particular affinity reagent, presence or absence of a post-translational modification, enzymatic activity, affinity for binding a particular protein or protein motif, or the like.

In some embodiments set forth herein, the term “vessel” can refer to an enclosure that contains a substance. The enclosure can be permanent or temporary with respect to the timeframe of a method set forth herein or with respect to one or more steps of a method set forth herein. Exemplary vessels include, but are not limited to, a well (e.g. in a multiwell plate or array of wells), test tube, channel, tubing, pipe, flow cell, bottle, vesicle, droplet that is immiscible in a surrounding fluid, or the like. A vessel can be entirely sealed to prevent fluid communication from inside to outside, and vice versa. Alternatively, a vessel can include one or more ingress (inlet) or egress (outlet) to allow fluid communication between the inside and outside of the vessel. A vessel can be made from multiple materials, for example, including a well in a solid support that is covered by a seal, such as a wax or fluid that is immiscible with a fluid in the well.

The embodiments set forth below and recited in the claims can be understood in view of the above definitions.

### Modulation of Binding Reagent Binding Specificity

In an aspect, provided herein is a method, comprising: a) detecting in a first fluidic medium the presence or absence of binding of binding reagents of a first plurality of binding reagents to a plurality of molecules, wherein the first fluidic medium has a first fluidic condition, b) detecting in a second fluidic medium the presence or absence of binding of binding reagents of a second plurality of binding reagents to the plurality of molecules, wherein the second fluidic medium has a second fluidic condition, wherein the first fluidic condition differs from the second fluidic condition, and c) characterizing an individual molecule of the plurality of molecules based upon a binding pattern of the binding reagents of the plurality of binding reagents to the plurality of molecules, wherein the binding pattern comprises a presence or absence of binding of a binding reagent of the plurality of binding reagents to the individual molecule for the first fluidic condition and the second fluidic condition. Preferably, the plurality of molecules may be provided in a single-molecule array format, thereby facilitating observation of the binding interactions of each individual molecule of the plurality of molecules with the binding reagents.

In another aspect, provided herein is a method, comprising: (a) detecting in a first fluidic medium the presence or absence of binding of binding reagents of a first plurality of binding reagents to a plurality of molecules, wherein the first fluidic medium has a first fluidic condition, (b) detecting in a second fluidic medium the presence or absence of binding of the binding reagents of a second plurality of binding reagents to the plurality of molecules, wherein the second fluidic medium has a second fluidic condition, wherein the first fluidic condition differs from the second fluidic condition, and (c) distinguishing a first molecule of the plurality of molecules from a second molecule of the plurality of molecules based upon a binding pattern of the binding reagents of the plurality of binding reagents to the plurality of molecules, wherein the binding pattern comprises for each individual molecule of the plurality of molecules a presence or absence of binding of a binding reagent of the plurality of binding reagents to the individual molecule for the first fluidic condition and the second fluidic condition.

A method set forth herein may occur in a vessel (e.g., a fluidic cartridge or flow cell) comprising a plurality of molecules. In some cases, a method that utilizes a first fluidic medium having a first fluidic condition and a second fluidic medium having a second fluidic condition can comprise withdrawing the first fluidic medium from a vessel and delivering the second fluidic medium to the vessel. Alternatively, a method may comprise altering a first fluidic medium having a first fluidic condition in a vessel, thereby forming the second fluidic medium having a second fluidic condition in the vessel.

In some cases, characterizing a molecule may comprise identifying the presence of a binding interaction between the molecule and a binding reagent. Characterizing the molecule may further comprise determining a binding strength of the binding interaction. Binding strength may be determined by any suitable method, including empirical correlations or in silico modeling of binding interactions in differing fluidic conditions. In some cases, characterizing a molecule may comprise determining an identity of the individual molecule. Methods of identifying analytes such as polypeptides based upon binding patterns or binding profiles is set forth herein. In cases in which a polypeptide is characterized, a method may comprise identifying an epitope of the polypeptide. In other cases, in which a polypeptide is characterized, a method may comprise identifying a proteoform (e.g., a post-translational modification, a splice variant, etc.) of the polypeptide.

In some cases, characterizing a molecule of a plurality of molecules can comprise individually characterizing two or more molecules of the plurality of molecules. In some cases, characterizing two or more molecules of the plurality of molecules can comprise characterizing each individual molecule of the plurality of molecules. In some cases, characterizing two or more molecules of a plurality of molecules can comprise quantifying a unique species of molecule in the sample based on a binding pattern that includes a first fluidic condition and a second fluidic condition. In some cases, characterizing two or more molecules of a plurality of molecules can comprise quantifying a proteoform of a unique species of molecule in the sample based on a binding pattern that includes a first fluidic condition and a second fluidic condition. In some cases, characterizing two or more molecules of a plurality of molecules can comprise quantifying two or more unique species of molecules in the sample based on a binding pattern that includes a first fluidic condition and a second fluidic condition. In some cases, characterizing two or more molecules of a plurality of molecules can comprise quantifying two or more proteoforms of a unique species of molecule in the sample based on a binding pattern that includes a first fluidic condition and a second fluidic condition. A method of quantifying one or more molecules of a plurality of molecules may be performed on a system set forth herein (e.g., a system comprising a processor that is configured to receive binding data and/or quantify one or more molecules).

Methods provided herein may be utilized for characterizing molecules from a known or characterized source. For example, a protein sample from a human can be expected to contain proteins from the human proteome. Alternatively, methods provided herein may be utilized for characterizing molecules from an unknown or uncharacterized source. For example, proteins derived from the effluent of a contaminated industrial device may be derived from an unknown organism. Accordingly, a method of characterizing one or more molecules that comprises identifying the one or more molecules based upon a binding pattern may comprise choosing a most likely molecular identity from amongst a database of candidate molecular identities. Likewise, a method of characterizing one or more molecules that comprises quantifying the one or more molecules based upon a binding pattern may comprise determining a quantity of molecules from a plurality of molecules that are likely to be a candidate molecule from a database of candidate molecules.

A binding interaction between a molecule and a binding reagent may comprise multiple phases, including at least two phases of: i) association between the molecule and the binding reagent, ii) existence of a complex containing the binding reagent and the molecule, and iii) dissociation of the binding reagent from the molecule. The association phase of a binding interaction between a molecule and a binding reagent may comprise the steps of: i) contacting the binding reagent to the molecule, optionally in the presence of a fluidic association medium, and ii) coupling the binding reagent to the molecule, thereby forming a complex containing the binding reagent and the molecule. The dissociation phase of a binding interaction between a molecule and a binding reagent may comprise the steps of: i) dissociating the binding reagent from the molecule, optionally in the presence of a fluidic dissociation medium, and ii) optionally separating the binding reagent from the molecule (e.g., diffusing the binding reagent away from the molecule, rinsing the binding reagent away from the molecule, magnetically or electrically withdrawing the binding reagent from the molecule, etc.).

A binding reagent may form a non-covalent binding interaction with a molecule. Formation of a non-covalent binding interaction may include each of the three phases of a binding interaction, as set forth herein. While a non-covalent complex between a binding reagent and a molecule exists, the complex may be detected, for example by detecting a fluorescent signal from the binding reagent. Alternatively, a non-covalent complex between a binding reagent and a molecule may be detected after dissociation of the complex, for example by detecting a chemical modification of the molecule that occurred due to the non-covalent binding of the binding reagent to the molecule (e.g., a chemical addition, cleavage, or substitution; transfer of a barcode from the binding reagent to the molecule, etc.). A binding reagent may form a covalent binding interaction with a molecule. For an irreversible covalent binding interaction, a dissociation phase of a binding interaction may not occur. For a reversible covalent binding interaction, a dissociation phase of a binding interaction may occur. A covalent complex between a binding reagent and a molecule may be detected, for example by detecting a fluorescent signal from the binding reagent.

An appropriate choice of binding reagent for a method set forth herein can depend upon the one or more molecules to be contacted with the binding reagent. Given that the methods set forth herein can be utilized to characterize both known and unknown characteristics of molecules, the choice of an appropriate binding reagent should not be limited. Binding reagents can comprise biomolecules and non-biological molecules. Biomolecules can comprise biopolymers (e.g., proteins, peptides, RNAs, DNAs, polysaccharides, etc.) or non-polymeric biomolecules (e.g., lipids, hormones, metabolites, etc.). Non-biological molecules can comprise synthetic molecules (e.g., synthetic polymers, microplastics, carbon particles, metal particles, semiconductor particles, mineral particles, ceramic particles, etc.) or engineered biomolecules (e.g., biopolymers substituted with one or more non-natural residues or moieties). A binding reagent can comprise a pharmaceutical compound (e.g., a synthetic pharmaceutical molecule, an engineered pharmaceutical molecule, a naturally-occurring pharmaceutical molecule, etc.), a toxin molecule (e.g., a naturally-occurring toxin, a synthetic or engineered toxin, etc.), a small molecule compound (i.e., a molecule having a molecular weight of less than 1 kiloDalton), or a macromolecule (i.e., a molecule having a molecular weight of 1 kiloDalton or more).

When characterizing molecules that are immobilized on a solid support (e.g., a single molecule array), a binding entity that has full degrees of spatial freedom during at least a portion of a characterization (e.g., before association of the binding reagent, after dissociation of the binding reagent) may be considered to be the binding reagent. For example, if a protein sample having protcome-scale protein diversity immobilized on a solid support is contacted with a fluidic medium containing antibodies, the antibodies would be considered the binding reagents. Conversely, if antibodies immobilized on a solid support are contacted with a fluidic medium containing a protein sample having proteome-scale protein diversity, the proteins of the protein sample may be considered to be the binding reagent.

Accordingly, observing the presence or absence of binding interactions between binding reagents and molecules may be performed during association, dissociation, or both, of the binding reagents to the molecules. For example, a method may comprise: a) detecting the presence or absence of binding of binding reagents of a first plurality of binding reagents to a plurality of molecules in a first fluidic association condition or a first fluidic dissociation condition, b) detecting the presence or absence of binding of the binding reagents of the first plurality of binding reagents to the plurality of molecules in a second fluidic association condition or a second fluidic dissociation conditions, in which the first fluidic association condition or first fluidic dissociation condition differs from the second fluidic association condition or second fluidic dissociation condition, and c) characterizing one or more molecules of the plurality of molecules based upon a binding pattern of the binding reagents of the plurality of binding reagents to the plurality of molecules, wherein the binding pattern comprises for each molecule of the plurality of molecules a presence or absence of binding of a binding reagent of the plurality of binding reagents for the first fluidic association or dissociation condition and the second fluidic association or dissociation condition.

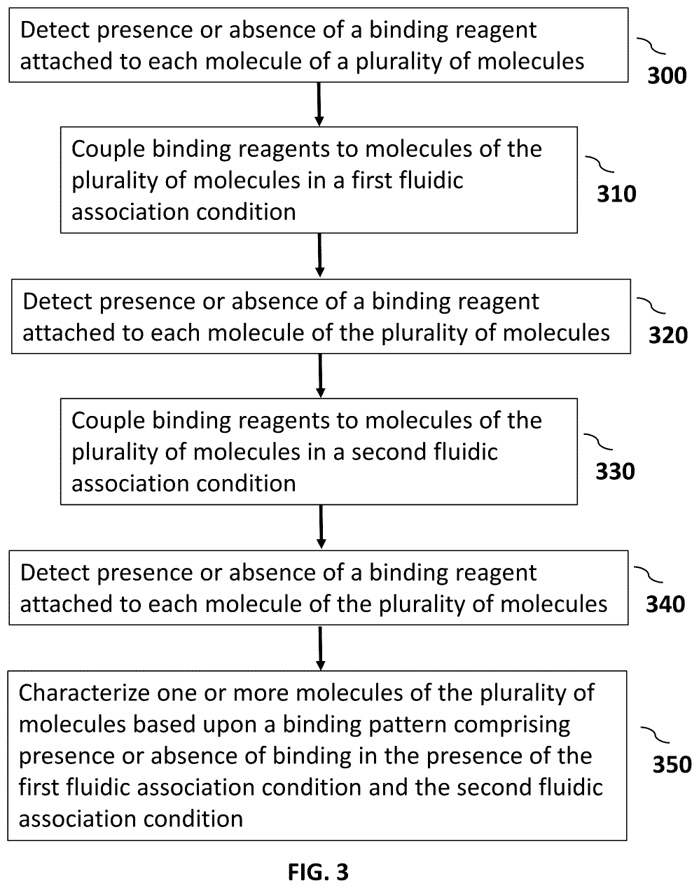

FIG. 3 depicts a flow chart for a method of characterizing one or more molecules utilizing two differing fluidic association conditions. In a first step 300, the presence or absence of a coupled binding reagent may be detected for each individual molecule of a plurality of molecules. In a second step 310, binding reagents may be coupled to molecules of the plurality of molecules in a first fluidic association condition. In a third step 320, the presence or absence of a coupled binding reagent may be detected for each individual molecule of the plurality of molecules. Optionally, after step 320, coupled binding reagents may be dissociated from the bound molecules of the plurality of molecules. In a fourth step 330, binding reagents may be coupled to molecules of the plurality of molecules in the presence of a second fluidic association condition. In a fifth step 340, the presence or absence of a coupled binding reagent may be detected for each individual molecule of the plurality of molecules. In a sixth step 350, one or more molecules of the plurality of molecules may be characterized based upon binding patterns for each individual molecule, wherein the binding pattern comprises the presence or absence of binding of the binding reagent to the individual molecule for the first fluidic association condition and the second fluidic association condition.

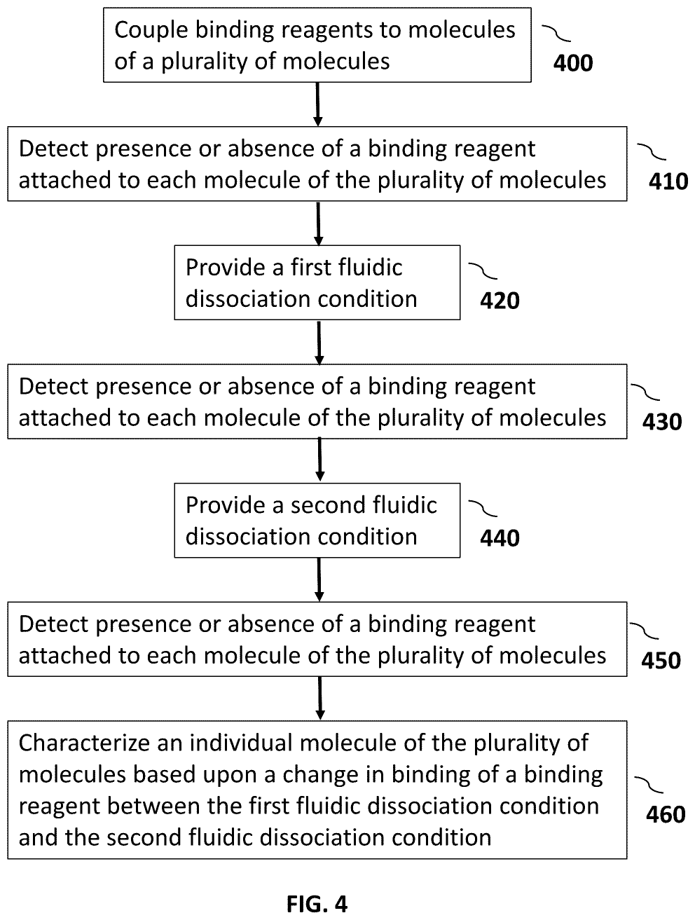

FIG. 4 depicts a flow chart for a method of characterizing an individual molecule utilizing two differing fluidic dissociation conditions. In a first step 400, binding reagents are coupled to molecules of a plurality of molecules. In a second step 410, the presence or absence of a binding reagent is detected for each individual molecule of the plurality of molecules. In a third step 420, a first fluidic dissociation condition is provided to the plurality of molecules. In a fourth step 430, the presence or absence of a binding reagent is detected for each individual molecule of the plurality of molecules. Optionally, after step 430, all bound binding reagents may be dissociated from molecules, and steps 400 and 410 may be repeated. In a fifth step 440, a second fluidic dissociation condition is provided to the plurality of molecules. In a sixth step 450, the presence or absence of a binding reagent is detected for each individual molecule of the plurality of molecules. In a seventh step 460, one or more molecules of the plurality of molecules may be characterized based upon binding patterns for each individual molecule, wherein the binding pattern comprises the presence or absence of binding of the binding reagent to the individual molecule for the first fluidic dissociation condition and the second fluidic dissociation condition.

The ability of a binding reagent to bind to a molecule may depend, at least in part, on the surrounding chemical environment. For binding interactions mediated by a fluid, the fluidic condition can affect the likelihood that a binding interaction will occur or be observed. For example, in the presence of a first fluidic condition, a binding reagent may bind tightly to a molecule, thereby facilitating observation of the complex formed between the molecule and the binding reagent. In the presence of a second fluidic condition, the binding reagent may bind weakly to a molecule, thereby increasing a likelihood that the binding reagent will dissociate from the molecule before the binding interaction can be observed. In another example, a binding reagent may readily react or catalyze a reaction with a molecule in the presence of a first fluidic condition. In the presence of a second fluidic condition, a binding reagent may be weakly reactive or catalytic with a molecule.

A fluidic condition can include any aspect of fluid formulation or properties that may affect the binding behavior of a binding reagent with a molecule. A fluidic condition may include aspects such as fluid composition, binding reagent concentration, fluid temperature, fluid pressure, fluid velocity, fluid depth or film thickness, magnetic field orientation or magnitude, and electrical field orientation or magnitude.

In an aspect, a method may comprise the steps of: a) detecting the presence or absence of binding of binding reagents of a first plurality of binding reagents to a plurality of molecules in the presence of a first fluidic composition, first fluidic temperature, first fluidic pressure, first velocity, first depth or film thickness, and/or first magnetic and/or electrical field orientation, b) detecting the presence or absence of binding of the binding reagents of the first plurality of binding reagents to the plurality of molecules in the presence of a second fluidic composition, second fluidic temperature, second fluidic pressure, second velocity, second depth or film thickness, and/or second magnetic and/or electrical field orientation, in which the first fluidic composition, fluidic temperature, fluidic pressure, velocity, depth or film thickness, and/or first magnetic and/or electrical field orientation, differs from the second fluidic composition, fluidic temperature, fluidic pressure, velocity, depth or film thickness, and/or magnetic and/or electrical field orientation, and c) characterizing one or more molecules of the plurality of molecules based upon a binding pattern of the binding reagents of the plurality of binding reagents to the plurality of molecules, wherein the binding pattern comprises for each molecule of the plurality of molecules a presence or absence of binding of a binding reagent of the plurality of binding reagents for the first fluidic composition, fluidic temperature, fluidic pressure, velocity, depth or film thickness, and/or first magnetic and/or electrical field orientation and the second fluidic composition, fluidic temperature, fluidic pressure, velocity, depth or film thickness, and/or first magnetic and/or electrical field orientation.

A fluid containing or contacted to a binding reagent may be provided at a temperature of at least about −80° C., −60° C., −40° C., −20° C., −10° C., −5° C., 0° C., 5° C., 10° C., 15° C., 16° C., 17° C., 18° C., 19° C., 20° C., 21° C., 22° C., 23° C., 24° C., 25° C., 30° C., 35° C., 40° C., 60° C., 80° C., 95° C., or more than 95° C. Alternatively or additionally, a fluid containing or contacted to a binding reagent may be provided at a temperature of no more than about 95° C., 80° C., 60° C., 40° C., 35° C., 30° C., 25° C., 24° C., 23° C., 22° C., 21° C., 20° C., 19° C., 18° C., 17° C., 16° C., 15° C., 10° C., 5° C., 0° C., −5° C., −10° C., −20° C., −40° C., −60° C., −80° C., or less than −80° C.

A method may comprise, in a first step, providing a fluidic medium containing or contacted to a binding reagent at a first temperature and, in a second step, providing the fluidic medium containing or contacted to the binding reagent at a second temperature, in which a temperature difference between the first temperature and the second temperature is at least about ±0.1° C., ±0.5° C., ±1° C., ±2° C., ±3° C., ±4° C., ±5° C., ±10° C., ±15° C., ±20° C., ±30° C., ±40° C., ±50° C., or more than ±50° C. Alternatively or additionally, a method may comprise, in a first step, providing a fluidic medium at a first temperature and, in a second step, providing the fluidic medium at a second temperature, in which a temperature difference between the first temperature and the second temperature is no more than about ±50° C., ±40° C., ±30° C., ±20° C., ±15° C., ±10° C., ±5° C., ±4° C., ±3° C., ±2° C., ±1° C., ±0.5° C., ±0.1° C., or less than ±0.1° C.

A fluid containing or contacted to a binding reagent may be provided at a pH of at least about 0, 0.5, 1.0, 1.5, 2.0, 2.5, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.5, 10.0, 10.5, 11.0, 11.5, 12.0, 12.5, 13.0, 13.5, 14.0, or more than 14.0. Alternatively or additionally, a fluid containing or contacted to a binding reagent may be provided at a pH of no more than about 14.0, 13.5, 13.0, 12.5, 12.0, 11.5, 11.0, 10.5, 10.0, 9.5, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.5, 2.0, 1.5, 1.0, 0.5, 0, or less than 0.

A method may comprise, in a first step, providing a fluidic medium containing or contacted to a binding reagent at a first pH and, in a second step, providing the fluidic medium containing or contacted to the binding reagent at a second pH, in which a pH difference between the first pH and the second pH is at least about ±0.1, ±0.5, ±1, ±1.5, ±2, ±2.5, ±3, ±3.5, ±4, ±4.5, ±5, ±10, or more than ±10. Alternatively or additionally, a method may comprise, in a first step, providing a fluidic medium containing or contacted to a binding reagent at a first pH and, in a second step, providing the fluidic medium containing or contacted to the binding reagent at a second pH, in which a pH difference between the first pH and the second pH is no more than about ±10, ±5, ±4.5, ±4, ±3.5, ±3, ±2.5, ±2, ±1.5, ±1, ±0.55, ±0.1, or less than ±0.1.

A fluidic medium contacted to a plurality of molecules and/or a plurality of binding reagents may be formulated to affect the likelihood or the strength of a binding interaction between a molecule of the plurality of molecules and a binding reagent of the plurality of binding reagents. For covalent interactions between a molecule and a binding reagent, a fluid composition contacted to the molecule and/or the binding reagent can affect the reactivity of one or both of the interaction partners. Reactivity may be affected by, for example, pH, ionic strength, and polarity of a fluidic medium. A fluidic medium may be formulated to increase or decrease a reactivity between a molecule and a binding reagent. A fluidic medium may be formulated to increase or decrease a reactivity between a first molecule and a binding reagent relative to the reactivity of a second molecule and the binding reagent. For non-covalent interactions between a molecule and a binding reagent, a fluid composition contacted to the molecule and/or the binding reagent can affect the binding strength of the binding interaction between the molecule and the binding reagent. Binding strength may be affected by, for example, pH, ionic strength, and polarity of a fluidic medium. A fluidic medium may be formulated to increase or decrease a binding strength between a molecule and a binding reagent. A fluidic medium may be formulated to increase or decrease a binding strength between a first molecule and a binding reagent relative to the binding strength of a second molecule and the binding reagent.

Useful reagents for formulation of a fluidic medium are set forth herein. A method may utilize two differing fluidic media, in which each individual fluidic medium differs with respect to chemical composition. Two or more fluidic media may differ with respect to one or more of: i) the presence, absence, or concentration of water, ii) the presence, absence, or concentration of a non-aqueous solvent, iii) the presence, absence, or concentration of a diluent, iv) the presence, absence, or concentration of an ionic compound, v) the presence, absence, or concentration of a buffering species, vi) the presence, absence, or concentration of a surfactant species, vii) the presence, absence, or concentration of a chaotropic species, viii) the presence, absence, or concentration of an acidifying agent, ix) the presence, absence, or concentration of an alkalizing agent, x) the presence, absence, or concentration of a blocking agent, xi) the presence, absence, or concentration of a kosmotropic species, xii) the presence, absence, or concentration of an excipient reagent, and xiii) the presence, absence, or concentration of a competitor or scavenger species. Useful formulations of fluidic media for facilitating binding interactions are described in U.S. Patent Publication No. 20240192202A1, which is herein incorporated by reference in its entirety.

Competitor or scavenger species may be provided to a fluidic medium that is contacted with a binding reagent and/or a molecule. A competitor species may be any binding reagent having a same or similar binding specificity as a binding reagent for one or more molecules. Binding of a competitor species to a molecule may inhibit binding of a binding reagent to the molecule. A competitor species may be substantially devoid of a detectable label, or may provide no detectable signal that facilitates the detection of the competitor species to a molecule. A competitor species may be a same species as a binding reagent (e.g., an antibody binding reagent and an antibody competitor, an aptamer binding reagent and an aptamer competitor, an enzymatic binding reagent and an enzymatic competitor, etc.). A competitor species may be a differing species from a binding reagent (e.g., an antibody binding reagent and an aptamer competitor, an aptamer binding reagent and an antibody competitor, an enzymatic binding reagent and a non-enzymatic competitor, etc.).

A scavenger species may be a molecule, particle, or moiety that can be bound by a binding reagent. In some cases, a scavenger species may have a common structural element as an epitope or moiety of a molecule that is bound by a binding reagent. For example, a binding reagent that couples to a trimer or tetramer amino acid sequence of a protein molecule may be scavenged by a peptide containing the trimer or tetramer amino acid sequence in a fluidic medium contacted to the binding reagent. In other cases, a scavenger species may not have a common structural element as an epitope or moiety of a molecule that is bound by a binding reagent. For example, an enzymatic binding reagent that modifies a protein molecule or a nucleic acid molecule may be scavenged by a small molecule that also binds to the active site of the enzyme.

Methods set forth herein may comprise detecting the presence or absence of a binding interaction between a binding reagent and a molecule of a plurality of molecules utilizing two or more different fluidic conditions. For each molecule of a plurality of molecules that is observed, a binding pattern for the molecule may be determined. For a single molecule of a plurality of molecules, a binding pattern may contain a quantitative or qualitative observation of the presence or absence of binding between the molecule and a binding reagent for each tested fluidic condition and/or binding reagent. For a plurality of molecules, a binding pattern can comprise a collection or aggregation of individual binding patterns for each molecule of the plurality of molecules. Qualitative observations could include binary or polynary classifications of the presence or absence of a binding interaction between an individual molecule and a binding reagent (e.g., bound/not bound/uncertain, etc.). Quantitative observations can depend upon the method of detection used to observe a binding interaction between an individual molecule and a binding reagent, and can include measures such as peak signal magnitude, average signal magnitude, signal-to-noise ratio, etc. Table I displays exemplary binding patterns for a presence or absence of binding of the individual molecule to a binding reagent. Based upon the data displayed in Table I, molecules 1 and 5 can be determined to have a same binding pattern for the tested fluidic conditions.

TABLE I

| | Fluidic Condition 1 | Fluidic Condition 2 |
|---|---|---|
| Molecule 1 | Bound | Bound |
| Molecule 2 | Bound | Not Bound |
| Molecule 3 | Not Bound | Not Bound |
| Molecule 4 | Not Bound | Bound |
| Molecule 5 | Bound | Bound |

A binding interaction between a molecule and a binding reagent may not be observed during a detection event, even when the molecule and binding reagent are able to form a complex. Failure to observe binding can occur due to variability in molecule conformation, variability in binding reagent conformation, and/or binding stochasticity. Accordingly, it may be useful to perform two or more observations of the presence or absence of a binding interaction between a molecule and a binding reagent. In some cases, performing two or more observations of the presence or absence of a binding interaction between a molecule and a binding reagent may comprise detecting the presence or absence of the binding interaction between the molecule and the binding reagent two or more times. In some cases, performing two or more observations of the presence or absence of a binding interaction between a molecule and a binding reagent may further comprise contacting the binding reagent to the molecule in the presence of the same fluidic condition for each of the two or more observations. If a molecule is observed to bind to a binding reagent at least once during two or more observations, it may be provided a classification of “bound,” for example. Further, a molecule may be classified according to the number of times it is observed to bind to a binding reagent. For example, if a molecule is observed to bind to a binding reagent once amongst four observations, it may be classified as “weakly binding.” Likewise, if the molecule is observed to bind to the binding reagent at least three times amongst the four observations, it may be classified as “strongly binding.”

An individual molecule of a plurality of molecules may be categorized according to its binding pattern. Categorizing molecules of a plurality of molecules may comprise determining one or more groups of one or more molecules having a same or similar binding profile. For example, in the data displayed in Table I, molecules 1 and 5 may be grouped into the same category due to each molecule individually having an identical binding pattern. In some cases, characterizing a molecule of a plurality of molecules may comprise one or more steps of: i) based upon a binding pattern of the molecule, determining a category for the molecule, and ii) based upon the category of the molecule, determining a characteristic of the molecule (e.g., an identity, a structural moiety, a structural motif, a modification, etc.).

A method may further comprise detecting the presence or absence of binding of a second binding reagent to a molecule under two differing fluidic conditions. Observations of binding interactions of a first binding reagent and a second binding reagent with a molecule or a plurality thereof may occur in a sequential fashion or a serial fashion. For example, binding interactions between a molecule and a first binding reagent may be observed in the presence of two differing fluidic conditions, then binding interactions between the molecule and a second binding reagent may be observed in the presence of two differing fluidic conditions. In some cases, binding interactions between a molecule and a first binding reagent may be observed in the presence of a first set of two differing fluidic conditions, and binding interactions between the molecule and a second binding reagent may be observed in the presence of a second set of two differing fluidic conditions, in which the first set of differing fluidic conditions and the second set of differing fluidic conditions are the same. In other cases, binding interactions between a molecule and a first binding reagent may be observed in the presence of a first set of two differing fluidic conditions, and binding interactions between the molecule and a second binding reagent may be observed in the presence of a second set of two differing fluidic conditions, in which the first set of differing fluidic conditions and the second set of differing fluidic conditions differ with respect to at least one fluidic condition. In other cases, binding interactions between a molecule and a first binding reagent may be observed in the presence of a first set of two differing fluidic conditions, and binding interactions between the molecule and a second binding reagent may be observed in the presence of a second set of two differing fluidic conditions, in which the first set of differing fluidic conditions and the second set of differing fluidic conditions are identical with respect to at least one fluidic condition.

A method may comprise the steps of: (a) detecting the presence or absence of binding of binding reagents of a first plurality of binding reagents to a plurality of molecules in the presence of a first fluidic condition, b) detecting the presence or absence of binding of binding reagents of a second plurality of binding reagents to the plurality of molecules in the presence of a second fluidic condition, in which the first fluidic condition differs from the second fluidic condition, and in which binding reagents of the first plurality of binding reagents differ (e.g., with respect to binding specificity) from binding reagents of the second plurality of binding reagents, and c) characterizing one or more molecules of the plurality of molecules based upon a binding pattern of the binding reagents of the plurality of binding reagents to the plurality of molecules. A binding pattern can comprise for each molecule of the plurality of molecules a presence or absence of binding of a binding reagent of the plurality of binding reagents for the first fluidic condition and the second fluidic condition.

A method may comprise the steps of: (a) providing a set of binding reagents, wherein the set of binding reagents comprises at least 3 (e.g., at least 4, 5, 10, 20, 25, 30, 40, 50, 100, 150, 200, 300, 400, 500, or more than 500) pluralities of binding reagents, wherein each plurality of binding reagents differs from every other plurality of binding reagents of the set of binding reagents, (b) for each plurality of binding reagents of the set of binding reagents, detecting the presence or absence of binding of binding reagents of the plurality of binding reagents to a plurality of molecules in the presence of a fluidic condition, wherein the fluidic conditions for the plurality of binding reagents differs from the fluidic condition of any other plurality of binding reagent of the set of binding reagents, and (c) characterizing one or more molecules of the plurality of molecules based upon a binding pattern of the binding reagents of the set of binding reagents to the plurality of molecules, wherein the binding pattern comprises for each molecule of the plurality of molecules a presence or absence of binding of each binding reagent of the set of binding reagents.

A binding pattern for a molecule may include binding observations from two or more binding reagents. Table II displays exemplary binding patterns for a plurality of individual molecules, in which the binding patterns contain qualitative observations of the presence or absence of binding of the individual molecule to the binding reagent observed in Table I, plus a second binding reagent. Based upon the data displayed in Table II, none of the molecules can be determined to have a same binding pattern for the tested fluidic conditions.

TABLE II

| | Binding Reagent 1 | | Binding Reagent 2 | |
|---|---|---|---|---|
| | Fluidic | Fluidic | Fluidic | Fluidic |
| | Condition 1 | Condition 2 | Condition 3 | Condition 4 |
| Molecule 1 | Bound | Bound | Not Bound | Not Bound |
| Molecule 2 | Bound | Not Bound | Bound | Not Bound |
| Molecule 3 | Not Bound | Not Bound | Not Bound | Not Bound |
| Molecule 4 | Not Bound | Bound | Bound | Bound |
| Molecule 5 | Bound | Bound | Bound | Not Bound |

A method may comprise detecting binding of a binding reagent to a molecule. In some cases, binding of a binding reagent to a molecule may be detected while a complex comprising the binding reagent and the molecule exists. In some cases, a molecule may be provided at a fixed address on a solid support, thereby facilitating observation of the presence or absence of binding of a binding reagent to the molecule by detection of the binding reagent at the fixed address. Accordingly, a binding reagent may comprise or be attached to a detectable label that facilitates detection of the binding reagent. Preferably, the detectable label may comprise a moiety that is configured to produce a detectable signal, such as a fluorescent moiety, a luminescent moiety, or a radiolabel. A signal from a detectable label may be detected by a sensor (e.g., a pixel-based array) that can spatially determine a location of the signal. In some cases, a method may comprise one or more steps of: i) providing a plurality of molecules immobilized on a solid support containing a plurality of addresses, wherein each individual address of the plurality of addresses contains one and only one molecule of the plurality of molecules, ii) coupling binding reagents to molecules of the plurality of molecules, wherein each binding reagent individually comprises a detectable label, and iii) detecting the presence or absence of a signal from a detectable label at each individual address of the plurality of addresses, thereby detecting the presence or absence of a binding reagent of the binding reagents bound to a molecule of the plurality of molecules at each individual address of the plurality of addresses.

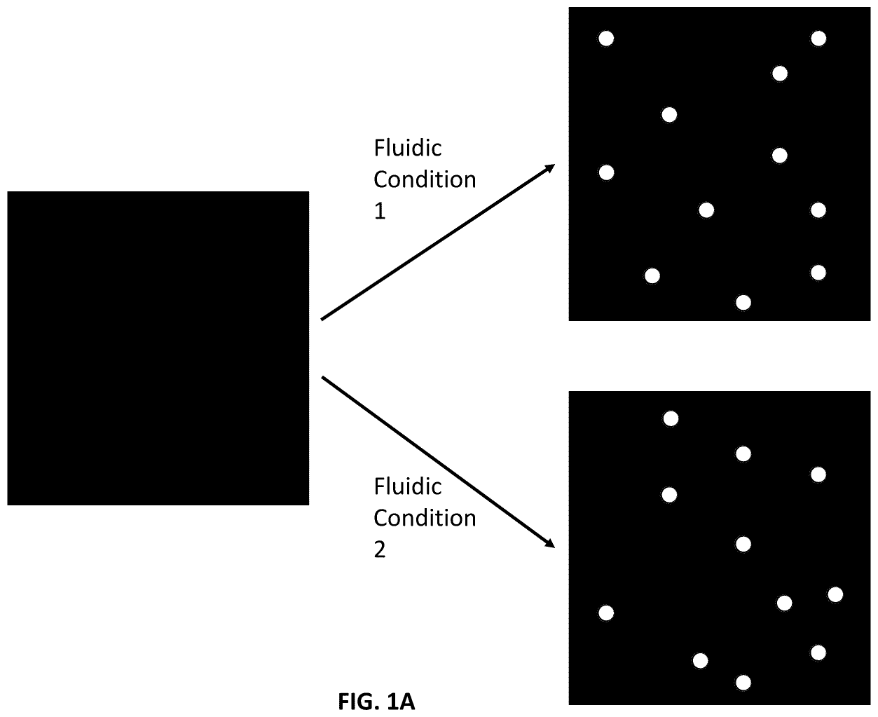

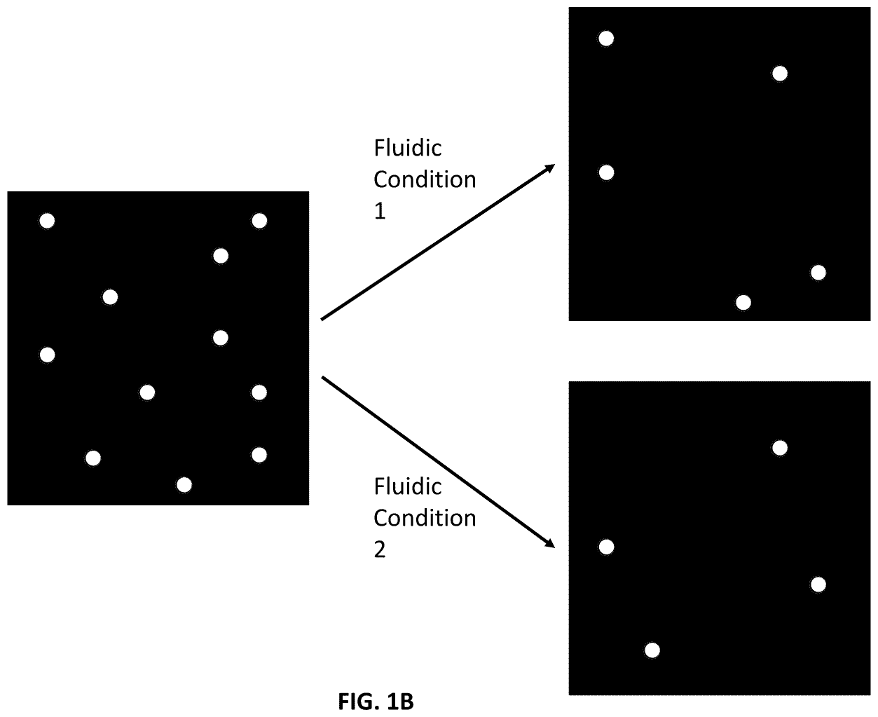

FIGS. 1A-1B depict aspects of characterizing molecules for association of binding reagents and dissociation of binding reagents, respectively. The left side of FIG. 1A depicts an image of a region of a solid support comprising a plurality of molecules before detectable binding reagents have been contacted to the solid support. No signals are observed at any addresses containing molecules. The right side of FIG. 1A depicts images of the region of the solid support after contacting the detectable binding reagents to the solid support in the presence of a first fluidic association condition (upper image) or a second fluidic association condition (lower image). Signals (depicted by white dots) are observed in each image corresponding to addresses containing molecules to which binding reagents are coupled. The observed spatial distribution of signals differs between the first fluidic association condition and the second fluidic association condition, thereby indicating different sets of molecules bound by each respective fluidic association condition. In the method depicted in FIG. 1A, binding reagents may be dissociated between detecting in the presence of the first fluidic association condition and detecting in the presence of the second fluidic association condition, thereby providing the unbound array depicted in the left side image. The left side of FIG. 1B depicts an image of a region of a solid support comprising a plurality of molecules after detectable binding reagents have been contacted to the solid support. Signals (depicted by white dots) are observed at a spatial distribution of addresses containing molecules bound by the detectable binding reagents. The right side of FIG. 1B depicts images of the region of the solid support after incubating the solid support in the presence of a first fluidic dissociation condition (upper image) or a second fluidic dissociation condition (lower image). Signals are observed in each image corresponding to addresses containing molecules to which binding reagents remain associated. The observed spatial distribution of signals differs between the first fluidic dissociation condition and the second fluidic dissociation condition, thereby indicating different sets of molecules dissociated from binding reagents by each respective fluidic dissociation condition. In the method depicted in FIG. 1B, binding reagents may be associated between detecting in the presence of the first fluidic dissociation condition and detecting in the presence of the second fluidic dissociation condition, thereby providing the bound array depicted in the left side image.

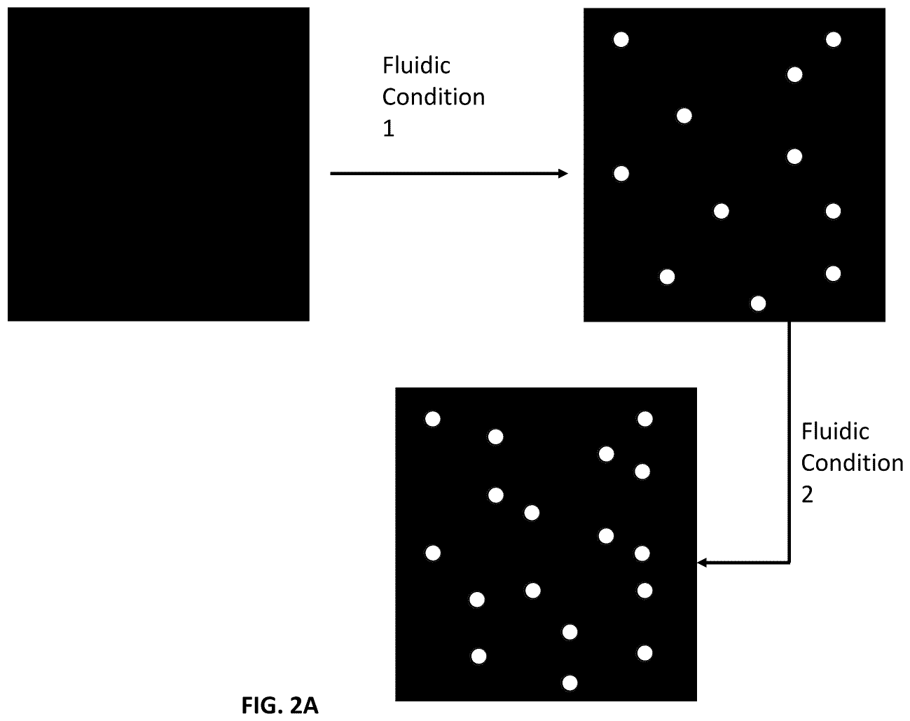

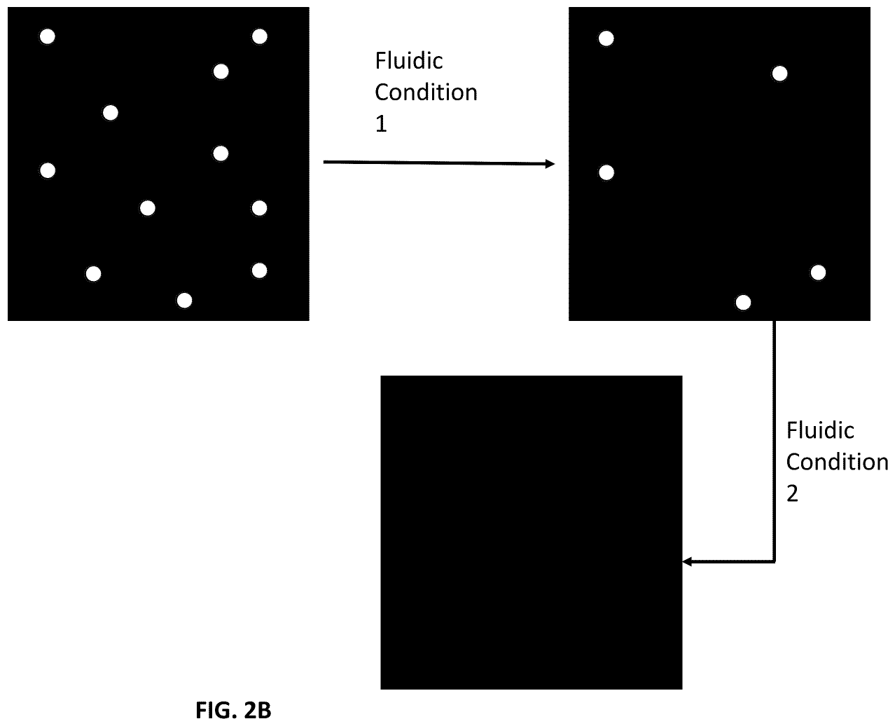

FIGS. 2A-2B depict aspects of characterizing molecules for association of binding reagents and dissociation of binding reagents, respectively, when differing fluidic conditions are provided to a solid support in a sequential manner. FIG. 2A depicts an image of a region of an unbound array. Binding reagents are contacted to the array in the presence of a first fluidic association condition. The top right image depicts observed signals from the region of the solid support after contacting the binding reagents in the presence of a first fluidic association condition. After detecting the signals of binding reagents bound to molecules in the presence of the first fluidic association condition, binding reagents are contacted to the array in the presence of a second fluidic association condition. The lower right image depicts observed signals from the region of the solid support after contacting the binding reagents in the presence of the second fluidic association condition. Comparing between the image after the first fluidic association condition and the image after the second fluidic association condition, differences may be observed in addresses containing associated binding reagents, corresponding to differing sets of molecules bound in the presence of each fluidic association condition. FIG. 2B depicts an image of a region of a binding reagent-bound array. The solid support is incubated in the presence of a first fluidic dissociation condition. The top right image depicts observed signals from the region of the solid support after incubating the binding reagents in the presence of a first fluidic dissociation condition. After detecting the signals of binding reagents bound to molecules in the presence of the first fluidic dissociation condition, binding reagents are incubated in the presence of a second fluidic dissociation condition. The lower right image depicts observed signals from the region of the solid support after incubating the binding reagents in the presence of the second fluidic dissociation condition. Comparing the image after the first fluidic dissociation condition and the image after the second fluidic dissociation condition, differences may be observed in addresses containing associated binding reagents, corresponding to differing sets of molecules dissociated from binding reagents in the presence of each fluidic dissociation condition.

Methods of the present disclosure may be performed at a binding equilibrium. An equilibrium binding method may comprise one or more steps of: (i) incubating a plurality of binding reagents with a plurality of molecules for a sufficient length of time to form a binding equilibrium between the plurality of binding reagents and the plurality of molecules (e.g., at least about 1 minute (min), 5 mins, 10 mins, 15 mins, 30 mins, or more than 30 mins), and (ii) detecting the presence or absence of binding of binding reagents to molecules of the plurality of molecules in the presence of unbound binding reagents. A system that is configured to facilitate detection at binding equilibrium may further facilitate providing a plurality of binding reagents under two differing fluidic conditions without a step of removing the plurality of binding reagents between the two differing fluidic conditions. For example, an equilibrium detection method may comprise the steps of: (i) detecting the presence or absence of binding of binding reagents to molecules of the plurality of molecules in the presence of unbound binding reagents at a first fluidic condition, (ii) altering the first fluidic condition to a second fluidic condition in the presence of the plurality of binding reagents and the plurality of molecules, and (iii) detecting the presence or absence of binding of binding reagents to molecules of the plurality of molecules in the presence of unbound binding reagents at the second fluidic condition.

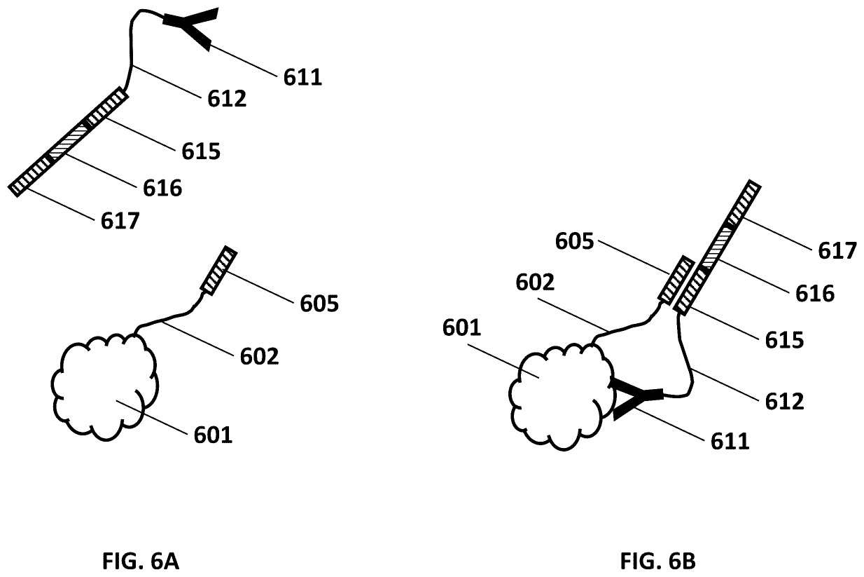

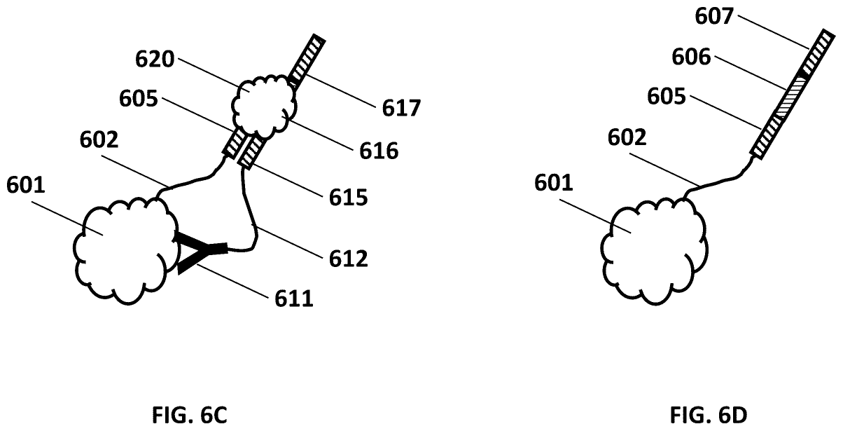

Binding of a binding reagent may be detected after a complex comprising the binding reagent and the molecule has dissociated. Methods comprising transfer of barcodes or tags (e.g., nucleic acid barcodes or tags, peptide barcodes or tags, etc.) may be useful for detecting the prior presence of binding of a binding reagent to a molecule after the binding reagent has dissociated from the molecule. FIGS. 6A-6D depicts a method of transferring a barcode to a molecule to detect the presence of a binding interaction. FIG. 6A depicts a molecule 601 that is attached to a nucleic acid first priming sequence 605 by an optional linker moiety 602 (e.g., a PEG molecule, an alkyl moiety, a nucleic acid linker, a peptide linker, etc.). The molecule 601 is contacted with a binding reagent 611, in which the binding agent is attached to a nucleic acid molecule by an optional linker 612. The nucleic acid molecule attached to the binding reagent 612 contains a first complementary priming sequence 615, a complementary barcode sequence 616, and a second complementary priming sequence 617. FIG. 6B depicts a configuration in which the binding reagent 611 has coupled to the molecule 601, thereby facilitating nucleic acid hybridization of the first complementary priming sequence 615 to the first priming sequence 605. FIG. 6C depicts a step in which a nucleic acid polymerase 620 binds to the nucleic acid complex, thereby transferring the barcode sequence to the nucleic acid molecule of the molecule 601. FIG. 6D depicts a configuration in which a barcode sequence 606 and a second priming sequence 607 have been attached to the first priming sequence 605. In some cases, a method of barcode transfer can be performed with a molecule that is immobilized on a solid support. If a molecule is immobilized on a solid support, a barcode may be attached to the solid support at the address containing the array or attached directly to the molecule. In other cases, a method of barcode transfer can be performed on a molecule that is not immobilized on a solid support (e.g., a molecule that is solubilized or suspended in a fluidic medium). A method of barcode transfer can be repeated under differing fluidic conditions, thereby transferring a cycle-specific barcode for each fluidic condition in which a binding reagent binds to the molecule.

A binding reagent may have a characterized binding specificity. A binding reagent may have a binding specificity for one and only one type or species of molecule. For example, an antibody or aptamer binding reagent may have a binding specificity for a single species or proteoform of protein. A binding reagent may have a binding specificity for more than one type or species of molecule. For example, an antibody or aptamer binding reagent may have a binding specificity for two or more species of protein or two or more proteoforms of a protein. In another example, a binding reagent may bind to two or more different proteins (e.g., different proteins as distinguished by primary amino acid sequence), in which each of the two or more different proteins comprises a same epitope. In some cases, a binding reagent may bind to a subset of a plurality of molecules. A subset may comprise two or more types of species of molecules (e.g., at least about 5, 10, 20, 50, 100, 200, 500, 1000, or more than 1000 types or species of molecules). Methods of selecting for and characterizing binding reagents that bind to more than one analyte are described in U.S. Pat. Nos. 11,970,693 and 11,993,865, each of which is herein incorporated by reference in its entirety. A method may comprise a step of detecting the presence or absence of binding of binding reagents of a plurality of binding reagents to a plurality of molecules in the presence of a fluidic condition, in which a molecule of the plurality of molecules has an unknown identity or an undetermined characteristic.

Methods of characterizing the binding specificities and/or affinities of binding reagents that are provided in, for example, U.S. Pat. Nos. 11,970,693 and 11,993,865, each of which is herein incorporated by reference in its entirety, may be readily extended to characterizing the binding reagents in two or more fluidic conditions. A binding model of a binding reagent may be provided, in which the binding model contains a binding probability for a binding reagent with each molecule of a plurality of different molecules in two or more fluidic conditions. The binding model may be provided to a processor that is configured to receive the binding model and binding data as measured by a method of the present disclosure. A method may further comprise characterizing one or more molecules of a plurality of molecules utilizing the binding model and the binding data.

Alternatively, a binding reagent may have an uncharacterized binding specificity. A method may comprise a step of detecting the presence or absence of binding of binding reagents of a plurality of binding reagents to a plurality of molecules in the presence of a fluidic condition, in which a binding reagent of the binding reagents has an unknown identity or an undetermined binding specificity. For example, a plurality of molecules comprising one or more types of molecules may be contacted with a plurality of pharmaceutical compounds with an uncharacterized binding specificity. Accordingly, determining a characteristic of one or more molecules of a plurality of molecules may comprise identifying one or more molecules that form a binding interaction with an uncharacterized binding reagent.

A method may utilize a first fluidic medium comprising a first binding reagent and a second fluidic medium comprising a second binding reagent, in which the first binding reagent is identical to the second binding reagent. A first binding reagent may be considered identical to a second binding reagent if the first binding reagent is indistinguishable from the second binding reagent. A method may comprise a step of altering a chemical or physical property of a first fluidic medium containing a first binding reagent, thereby forming a second fluidic medium comprising the first binding reagent. A method may utilize a first fluidic medium comprising a first plurality of binding reagents and a second fluidic medium comprising a second plurality of binding reagents, in which the first plurality of binding reagents is identical to the second plurality of binding reagents. A first plurality of binding reagents may be considered identical to a second plurality of binding reagents if the binding reagents of the first plurality of binding reagents are indistinguishable from the binding reagents of the second plurality of binding reagents. A method may comprise a step of altering a chemical or physical property of a first fluidic medium containing a first plurality of binding reagents, thereby forming a second fluidic medium comprising the first plurality of binding reagents.

Methods set forth herein may be readily provided in a multiplex format. A method may comprise providing a plurality of binding reagents comprising a first binding reagent or a plurality thereof and a second binding reagent or a plurality thereof, in which the first binding reagent and the second binding reagent have different binding specificities. A method may comprise the steps of: a) detecting the presence or absence of binding of binding reagents of a first plurality of binding reagents to a plurality of molecules in the presence of a first fluidic condition, and detecting the presence or absence of binding of binding reagents of a second plurality of binding reagents to the plurality of molecules in the presence of the first fluidic condition, b) detecting the presence or absence of binding of binding reagents of the first plurality of binding reagents to the plurality of molecules in the presence of a second fluidic condition, and detecting the presence or absence of binding of binding reagents of the second plurality of binding reagents to the plurality of molecules in the presence of the second fluidic condition, and c) characterizing one or more molecules of the plurality of molecules based upon a binding pattern of the binding reagents of the first plurality of binding reagents and the binding reagents of the second plurality of binding reagents to the plurality of molecules between the first fluidic condition and the second fluidic condition.

In another aspect, provided herein is a method, comprising: a) detecting the presence or absence of binding of binding reagents of a plurality of binding reagents to a plurality of molecules in the presence of a first fluidic condition, wherein the plurality of binding reagents comprises a first set of binding reagents and a second set of binding reagents, wherein binding reagents of the first set of binding reagents bind to a first set of molecules of the plurality of molecules, and wherein binding reagents of the second set of binding reagents bind to a second set of molecules of the plurality of molecules, b) detecting the presence or absence of binding of binding reagents of the plurality of binding reagents to the plurality of molecules in the presence of a second fluidic condition, and c) characterizing an individual molecule of the plurality of molecules based upon a binding pattern of the binding reagents of the plurality of binding reagents to the plurality of molecules between the first fluidic condition and the second fluidic condition.

A plurality of binding reagents may be provided to a method set forth herein, in which the plurality of binding reagents comprises a first binding reagent and a second binding reagent, and in which the first binding reagent and the second binding reagent have differing binding specificities. A first binding reagent and a second binding reagent may be divergent with respect to binding specificity. The binding specificity of a first binding reagent may be divergent from the binding specificity of a second binding reagent if a set of molecules bound by the first binding reagent is distinguishable from a set of molecules bound by the second binding reagent. For example, a first antibody may have a divergent binding specificity to a second antibody if the first antibody is characterized as binding to methylated proteoforms of a protein and the second antibody is characterized as binding to phosphorylated proteoforms. In another example, a first antibody may have a divergent binding specificity to a second antibody if the first antibody may be characterized as binding to any protein containing an epitope having amino acid sequence DTX (where X is arginine, histidine, or lysine) and a second antibody may be characterized as binding to any protein containing an epitope having amino acid sequence HSZ (where Z is asparagine, glutamine, or proline). In some cases, a first binding reagent may have a divergent binding specificity to a second binding reagent if a set of molecules bound by the first binding reagent and a set of molecules bound by the second binding reagent contain no molecules common to both sets of molecules (i.e., orthogonal binding specificity). In other cases, a first binding reagent may have a divergent binding specificity to a second binding reagent if a set of molecules bound by the first binding reagent and a set of molecules bound by the second binding reagent each contain at least one molecule that is not common to both sets of molecules.

A first binding reagent and a second binding reagent may be divergent with respect to dissociation specificity. The dissociation specificity of a first binding reagent may be divergent from the dissociation specificity of a second binding reagent if a set of molecules dissociated from the first binding reagent is distinguishable from a set of molecules dissociated from the second binding reagent in the presence of a dissociation condition. For example, a first binding reagent may dissociate from molecules in the presence of a fluidic medium containing a surfactant, and a second binding reagent may not dissociate from molecules in the presence of the fluidic medium containing the surfactant. Accordingly, molecules bound by the first binding reagent may be distinguished from molecules bound by the second binding reagent if dissociation is observed in the presence of the fluidic medium containing the surfactant.

Divergent binding specificity or orthogonal dissociation specificity may include divergence with respect to association or dissociation conditions, respectively. For example, a first binding reagent and a second binding reagent may have divergent binding or dissociation specificities in each of a first fluidic condition and a second fluidic condition. In another example, a first binding reagent and a second binding reagent may have divergent binding or dissociation specificities in a first fluidic condition and identical binding or dissociation specificities in a second fluidic condition.

Binding specificities of binding reagents may be characterized in a probabilistic fashion. A binding reagent may be characterized as having a probability of binding to any molecule or epitope or structure thereof. A first binding reagent may have an orthogonal binding specificity to a second binding reagent if the first binding reagent has a likelihood of binding a molecule or an epitope or moiety thereof above an upper threshold probability (e.g., at least about 90%, 95%, 99%, 99.9%, 99.99%, 99.999%, 99.9999%, 99.99999%, 99.999999%, etc.) and the second binding reagent has a likelihood of binding the molecule or the epitope or moiety thereof below a lower threshold probability (e.g., no more than 10%, 5%, 1%, 0.1%, 0.01%, 0.001%, 0.0001%, 0.00001%, 0.000001%, 0.0000001%, etc.). A first binding reagent may have a divergent binding specificity to a second binding reagent if there exists a set of two or more molecules for which the first binding reagent has a likelihood of individually binding each molecule of the set above an upper threshold probability (e.g., at least about 90%, 95%, 99%, 99.9%, 99.99%, 99.999%, 99.9999%, 99.99999%, 99.999999%, etc.) and the second binding reagent has a likelihood of individually binding each molecule of the set below a lower threshold probability (e.g., no more than 10%, 5%, 1%, 0.1%, 0.01%, 0.001%, 0.0001%, 0.00001%, 0.000001%, 0.0000001%, etc.). Analogously, dissociation specificities may be characterized in a probabilistic fashion.

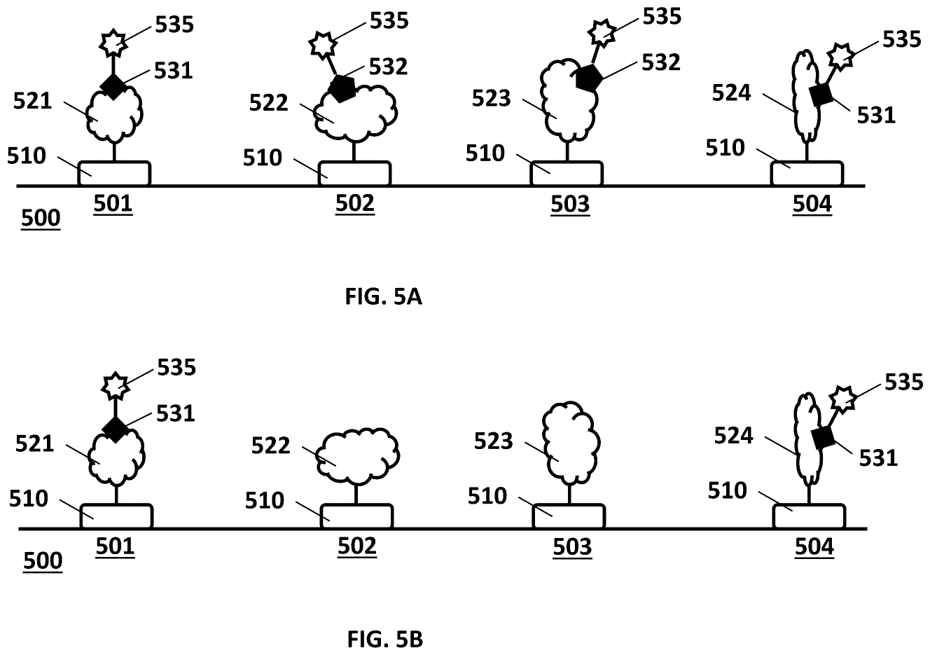

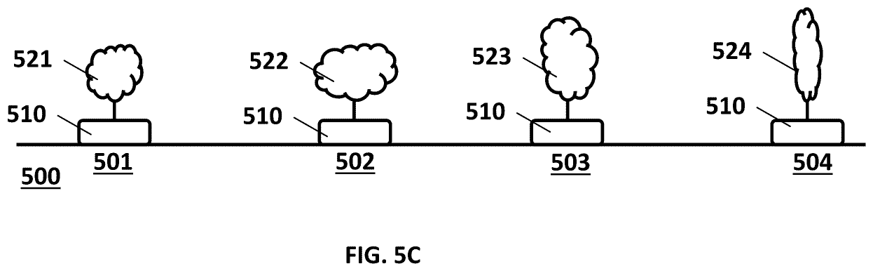

If a first binding reagent and a second binding reagent have divergent binding or dissociation specificities, the first binding reagent and the second binding reagent may each be individually provided with an identical detectable label (e.g., identical fluorophores). FIGS. 5A-5C depict aspects of a method of characterizing molecules utilizing divergent binding reagents with a single detectable label. FIG. 5A depicts a solid support 500 containing four addresses (501, 502, 503, and 504). Addresses 501, 502, 503, and 504 each individually contain a single molecule (521, 522, 523, and 524, respectively). Each single molecule is immobilized at an address by an anchoring moiety that is attached to the single molecule. Molecules 521 and 524 are bound to first binding reagents 531. Molecules 522 and 523 are bound to second binding reagents 532. Each individual first binding reagent 531 and each individual second binding reagent 532 is individually attached to a detectable label 535. FIG. 5B depicts a configuration of the solid support 500 after the solid support 500 has been incubated with a first fluidic dissociation medium. Molecules 522 and 523 have dissociated from second binding reagents 532, while molecules 521 and 524 remain bound to first binding reagents 531. Accordingly, a signal from detectable labels 535 will only be detected at addresses 501 and 504. FIG. 5C depicts a configuration of the solid support 500 after the solid support 500 has been incubated with a second fluidic dissociation medium. Molecules 521 and 524 have dissociated from the first binding reagents 531. Accordingly, molecules 521 and 524 may be characterized as distinct from molecules 522 and 523 due to the divergent dissociation specificities of the first binding reagents 531 and the second binding reagents 532.

The present disclosure also provides systems that are configured to implement methods set forth herein. In another aspect, provided herein is a system for analyte characterization, comprising: (a) a vessel comprising a plurality of molecules, (b) a binding reagent kit comprising a plurality of vessels, wherein each vessel of the plurality of vessels comprises a fluidic medium comprising a plurality of binding reagents, (c) a fluidic system that is configured to transfer the fluidic medium comprising the plurality of binding reagents to the vessel comprising the plurality of molecules, and (d) a detection system that is configured to detect at single-analyte resolution for each individual molecule of the plurality of molecules a presence or absence of binding of a binding reagent of the plurality of binding reagents. A system may comprise a vessel comprising a plurality of molecules, in which the molecules of the plurality of molecules are immobilized within the vessel (e.g., immobilized on an array, as set forth herein).

A system may further comprise a processor, wherein the processor is configured to: (i) receive detection data from the detection system, (ii) form a binding pattern from the detection data, wherein the binding pattern comprises for each individual molecule of the plurality of molecules a presence or absence of binding of a binding reagent of each plurality of binding reagents to the individual molecule for a first fluidic condition and a second fluidic condition, and (iii) based upon the binding pattern, characterize one or more molecules of the plurality of molecules. Alternatively or additionally, a system may comprise a processor, wherein the processor is configured to: (i) receive detection data from the detection system, (ii) form a binding pattern from the detection data, wherein the binding pattern comprises for each individual molecule of the plurality of molecules a presence or absence of binding of a binding reagent of each plurality of binding reagents to the individual molecule for a first fluidic condition and a second fluidic condition, and (iii) based upon the binding pattern, distinguish a first molecule of the plurality of molecules from a second molecule of the plurality of molecules.

In some cases, a system may further comprise a first processor, wherein the detection system further comprises a second processor, and wherein the second processor is configured to: (i) receive detection data, and (ii) form a binding pattern from the detection data, wherein the binding pattern comprises for each individual molecule of the plurality of molecules a presence or absence of binding of a binding reagent of each plurality of binding reagents to the individual molecule for a first fluidic condition and a second fluidic condition. Accordingly, the first processor may be configured to: (i) receive a binding pattern from the second processor, and (ii) based upon the binding pattern, characterize one or more molecules of the plurality of molecules. Alternatively or additionally, the first processor may be configured to: (i) receive a binding pattern from the second processor, and (ii) based upon the binding pattern, distinguish a first molecule of the plurality of molecules from a second molecule of the plurality of molecules.

A system may be configured to alter a first fluidic medium, thereby forming a second fluidic medium. A first fluidic medium may be altered in a vessel comprising a plurality of molecules or analytes, as set forth herein. In some configurations, a fluidic system may be configured to deliver a fluid to a vessel comprising a first fluidic medium, thereby forming a second fluidic medium. For example, a fluidic system may deliver a fluid comprising a diluent, solvent, co-solvent, ionic species, surfactant, blocking agent, acidifying agent, alkalizing agent, kosmotropic species, chelating species, excipient agent, or a combination thereof to a vessel comprising a first fluidic medium, thereby forming a second fluidic medium. Accordingly, a system may comprise one or more vessels in fluidic communication with a fluidic system, wherein the one or more vessels comprise a fluid that is substantially devoid of binding reagents (e.g., a fluid comprising a diluent, solvent, co-solvent, ionic species, etc.). In other configurations, a fluidic system may comprise a second vessel, wherein the fluidics system is configured to: (i) withdraw a first fluidic medium or a portion thereof from a first vessel comprising a plurality of molecules, (ii) deliver the first fluidic medium or the portion thereof to the second vessel, (iii) deliver a fluid to the second vessel, and (iv) after combining the first fluidic medium or the portion thereof with the fluid in the second vessel, thereby forming the second fluidic medium, delivering the second fluidic medium to the first vessel.

A system may comprise a device that is configured to alter a physical property of a fluidic medium (e.g., alter a temperature, pressure, velocity, depth or film thickness, magnetic field, or electrical field of a fluidic medium). A device may be configured to alter a physical property of a fluidic medium in a vessel comprising the fluidic medium. Alternatively, a device may be incorporated with a fluidic system, thereby facilitating alteration of the physical property of the fluidic medium before delivering the fluidic medium to a vessel. Exemplary devices can include heating/cooling devices (Pelletier devices, heat exchangers, etc.), pumps, magnets, and electromagnets.

A system may comprise a fluidic system that is configured to transfer fluids from a binding reagent kit, as set forth herein. A fluidic system may be configured to deliver a first fluidic medium comprising a first plurality of binding reagents and a second fluidic medium comprising a second plurality of binding reagents to a vessel, in which the first fluidic medium is withdrawn from a first vessel of a binding reagent kit and the second fluidic medium is withdrawn from a second vessel of the binding reagent kit. Alternatively, a fluidics system may be configured to withdraw from a vessel of a binding reagent kit a first volume of fluid comprising a first plurality of binding reagents and a second volume of fluid comprising a second plurality of binding reagents. The first volume of fluid may be equal to the second volume of fluid, thereby providing equal quantities of binding reagents to each volume of fluid. Alternatively, the first volume of fluid may be greater or less than the second volume of fluid. The first volume, second volume, or both may be mixed with a fluid that is substantially devoid of binding reagents, thereby providing differing fluidic conditions to the first volume and the second volume.

The present disclosure also provides kits comprising multiple binding reagent compositions. Each kit may comprise a plurality of vessels, each vessel comprising a binding reagent composition. A binding reagent composition can comprise a plurality of binding reagents solubilized or suspended in a fluidic medium. A binding reagent kit may comprise at least about 2, 5, 10, 20, 30, 40, 50, 60, 70, 80, 90, 100, 120, 150, 200, 250, 300, 400, 500, 1000, or more than 1000 vessels, wherein each vessel comprises a binding reagent composition. Alternatively or additionally, a binding reagent kit may comprise no more than about 1000, 500, 400, 300, 250, 200, 150, 120, 100, 90, 80, 70, 60, 50, 40, 30, 20, 10, 5, or less than 5 vessels, wherein each vessel comprises a binding reagent composition.

In an aspect, provided herein is a kit comprising a plurality of vessels (e.g., a well-based plate, etc.), wherein each vessel of the plurality of vessels comprises a fluidic medium, wherein the fluidic medium comprises a plurality of binding reagents, and wherein the kit is further characterized by at least one of (e.g., at least two of, at least three of, or each of): (a) a vessel comprising a first plurality of binding reagents in a first fluidic medium and a different vessel comprising a second plurality of binding reagents in a second fluidic medium, wherein the first plurality of binding reagents is identical to the second plurality of binding reagents, and wherein the first fluidic medium differs from the second fluidic medium, (b) a vessel comprising a first plurality of binding reagents in a first fluidic medium and a different vessel comprising a second plurality of binding reagents in a second fluidic medium, wherein a concentration of the first plurality of binding reagents in the first fluidic medium differs from a concentration of the second plurality of binding reagents in the second fluidic medium, and wherein the first fluidic medium is identical to the second fluidic medium, (c) a vessel comprising a first plurality of binding reagents in a first fluidic medium and a different vessel comprising a second plurality of binding reagents in a second fluidic medium, wherein the first plurality of binding reagents differs from the second plurality of binding reagents, and wherein the first fluidic medium differs from the second fluidic medium, and (d) a vessel comprising a first plurality of binding reagents in a first fluidic medium and a different vessel comprising a second fluidic medium, wherein the second fluidic medium is substantially devoid of binding reagents.

In another aspect, provided herein is a kit comprising at least 20 vessels, wherein each vessel of the at least 20 vessels comprises a fluidic medium, wherein the fluidic medium comprises a plurality of binding reagents, and wherein the kit is further characterized by at least one of (e.g., at least two of, or each of): (a) a vessel of the at least 20 vessels comprising a first plurality of binding reagents in a first fluidic medium and a different vessel of the at least 20 vessels comprising a second plurality of binding reagents in a second fluidic medium, wherein the first plurality of binding reagents is identical to the second plurality of binding reagents, and wherein the first fluidic medium differs from the second fluidic medium, (b) a vessel of the at least 20 vessels comprising a first plurality of binding reagents in a first fluidic medium and a different vessel of the at least 20 vessels comprising a second plurality of binding reagents in a second fluidic medium, wherein a concentration of the first plurality of binding reagents in the first fluidic medium differs from a concentration of the second plurality of binding reagents in the second fluidic medium, and wherein the first fluidic medium is identical to the second fluidic medium, and (c) at least 10 vessels containing 10 differing pluralities of binding reagents, wherein each of the 10 differing pluralities of binding reagents differs from each other plurality of binding reagents of the at least 10 differing pluralities of binding reagents with respect to binding reagent structure, binding specificity, or concentration.

In some cases, a binding reagent kit may further comprise one or more vessels comprising a fluidic medium, wherein the fluidic medium is substantially devoid of binding reagents, and wherein the fluidic medium comprises a diluent, a solvent, a co-solvent, an ionic compound, a buffering species, a surfactant species, a chaotropic species, an acidifying agent, an alkalizing agent, a blocking agent, a kosmotropic species, an excipient reagent, or a combination thereof.

A binding reagent kit may comprise a first vessel comprising a first plurality of binding reagents and a second vessel comprising a second plurality of binding reagents, wherein a binding reagent of the first plurality of binding reagents is identical to a binding reagent of the second plurality of binding reagents. A binding reagent kit may comprise a first vessel comprising a first plurality of binding reagents and a second vessel comprising a second plurality of binding reagents, wherein the first plurality of binding reagents is substantially identical to the second plurality of binding reagents. A binding reagent kit may comprise a first vessel comprising a first plurality of binding reagents and a second vessel comprising a second plurality of binding reagents, wherein a binding reagent of the first plurality of binding reagents is identical to a binding reagent of the second plurality of binding reagents, and wherein the first plurality of binding reagents differs from the second plurality of binding reagents (e.g., two different multiplexed mixtures of binding reagents containing a common binding reagent).

A binding reagent kit may comprise at least about 10 vessels, in which each of the at least about 10 vessels comprises a different binding reagent composition. A binding reagent kit may comprise at least about 2, 5, 10, 15, 20, 25, 30, 40, 50, 100, 120, 150, 200, 250, 300, 400, 500, 1000, or more than 1000 different binding reagent compositions. Alternatively or additionally, a binding reagent kit may comprise no more than about 1000, 500, 400, 300, 250, 200, 150, 120, 100, 50, 40, 30, 25, 20, 15, 10, 5, or fewer than 5 binding reagent compositions. A binding reagent composition may differ from any other binding reagent composition of a binding reagent kit with respect to binding reagent structure, binding specificity, binding reagent concentration, or fluidic conditions. A binding reagent kit may comprise at least 2 binding reagent compositions that are substantially identical.

Also provided herein is a method of utilizing a binding reagent kit, as set forth herein, comprising: (a) serially delivering each binding reagent composition or a subset thereof of a plurality of binding reagent compositions of the binding reagent kit to a vessel comprising a plurality of molecules, (b) for each binding reagent composition delivered to the vessel, detecting the presence or absence of binding of binding reagents of the binding reagent composition to molecules of the plurality of molecules, and (c) characterizing one or more molecules of the plurality of molecules based upon a binding pattern of binding reagents to the one or more molecules of the plurality of molecules, wherein the binding pattern comprises for each binding reagent composition delivered to the vessel a presence or absence of binding of a binding reagent of the binding reagent composition to the one or more molecules. The method may be performed on a system, as set forth herein.

Also provided herein is a method of utilizing a binding reagent kit, as set forth herein, comprising: (a) serially delivering each binding reagent composition or a subset thereof of a plurality of binding reagent compositions of the binding reagent kit to a vessel comprising a plurality of molecules, (b) for each binding reagent composition delivered to the vessel, detecting the presence or absence of binding of binding reagents of the binding reagent composition to molecules of the plurality of molecules, and (c) distinguishing a first molecule of the plurality of molecules from a second molecule of the plurality of molecules based upon a binding pattern of binding reagents to the first molecule and the second molecule of the plurality of molecules, wherein the binding pattern comprises for each binding reagent composition delivered to the vessel a presence or absence of binding of a binding reagent of the binding reagent composition to the first molecule and the second molecule.

### Single-Analyte Assays

The present disclosure provides compositions, apparatus and methods for detecting one or more proteins. A protein can be detected using one or more affinity agents having binding affinity for the protein. The affinity agent and the protein can bind each other to form a complex and, during or after formation, the complex can be detected. The complex can be detected directly, for example, due to a label that is present on the affinity agent or protein. In some configurations, the complex need not be directly detected. For example, complex formation can yield a chemical change, such as the formation of a nucleic acid tag, that is detected after the complex has been formed and, in some cases, after the complex has been dissociated.

The present disclosure provides compositions, apparatus and methods that can be useful for characterizing analytes, such as proteins, by obtaining multiple separate and non-identical measurements of the analytes. In particular configurations, the individual measurements may not, by themselves, be sufficiently accurate or specific to make the characterization, but in combination, the multiple non-identical measurements can allow the characterization to be made with a high degree of accuracy, specificity and confidence. For example, the multiple separate measurements can include subjecting a sample to reagents that are promiscuous with regard to recognizing a variety of different analytes that are present in the sample. Accordingly, a first measurement carried out using a first promiscuous reagent may perceive a first subset of the analytes without distinguishing different analytes within the subset. A second measurement carried out using a second promiscuous reagent may perceive a second subset of analytes, again, without distinguishing one analyte in the second subset from other analytes in the second subset. However, a comparison of the first and second measurements can distinguish: (i) an analyte that is uniquely present in the first subset but not the second; (ii) an analyte that is uniquely present in the second subset but not the first; (iii) an analyte that is uniquely present in both the first and second subsets; or (iv) an analyte that is uniquely absent in the first and second subsets. The number of promiscuous reagents used, the number of separate measurements acquired, and the degree of reagent promiscuity (e.g. the diversity of components recognized by the reagent) can be adjusted to suit the diversity of analytes expected for a particular sample.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as cells, organelles, nucleic acids, polysaccharides, metabolites, vitamins, hormones, enzyme co-factors, therapeutic agents, candidate therapeutic agents and others set forth herein or known in the art. Particular configurations of the methods, apparatus and compositions set forth herein can be made and used, for example, as set forth in U.S. Pat. No. 10,473,654 or 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g. amount or concentration), chemical reactivity, molecular structure, structural integrity (e.g. full length or fragmented), maturation state (e.g. presence or absence of pre- or pro-sequence in a protein), location (e.g. in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g. amino acid sequence length, overall charge or overall pKa for a protein) or common moiety (e.g. a short primary sequence motif or post-translational modification for a protein). An analyte can be characterized with regard to a relatively specific characteristic such as a unique amino acid sequence (e.g. for the full length of the protein or a motif), an RNA or DNA sequence that encodes a protein (e.g. for the full length of the protein or a motif), or an enzymatic or other activity that identifies a protein. A characterization can be sufficiently specific to identify an analyte, for example, at a level that is considered adequate or unambiguous by those skilled in the art.

In particular configurations, a method set forth herein can be used to identify a number of different extant proteins that exceeds the number of affinity reagents used. For example, the number of different protein species identified can be at least 5×, 10×, 25×, 50×, 100× or more than the number of affinity reagents used. This can be achieved, for example, by (1) using promiscuous affinity reagents that bind to multiple different candidate proteins suspected of being present in a given sample, and (2) subjecting the extant proteins to a set of promiscuous affinity reagents that, taken as a whole, are expected to bind each candidate protein in a different combination, such that each candidate protein is expected to generate a unique profile of binding and non-binding events when subjected to the set. Promiscuity of an affinity reagent can arise due to the affinity reagent recognizing an epitope that is known to be present in a plurality of different candidate proteins. For example, epitopes having relatively short amino acid lengths such as dimers, trimers, tetramers or pentamers are expected to occur in a substantial number of different proteins in a typical proteome. Alternatively or additionally, a given promiscuous affinity reagent may recognize multiple different epitopes (e.g. epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope.

Although performing a single binding reaction between a promiscuous affinity reagent and a complex protein sample may yield ambiguous results regarding the identity of the different extant proteins to which it binds, the ambiguity can be resolved by decoding the binding profiles for each extant protein using machine learning or artificial intelligence algorithms that are based on probabilities for the affinity reagents binding to candidate proteins. For example, a plurality of different promiscuous affinity reagents can be contacted with a complex population of extant proteins, wherein the plurality is configured to produce a different binding profile for each candidate protein suspected of being present in the population. The plurality of promiscuous affinity reagents can produce a binding profile for each extant protein that can be decoded to identify a unique combination of positive outcomes (i.e. observed binding events) and/or negative binding outcomes (i.e. observed non-binding events), and this can in turn be used to identify the extant protein as a particular candidate protein having a high likelihood of exhibiting a similar binding profile.

Binding profiles can be obtained for extant proteins and the binding profiles can be decoded or disambiguated to identify extant proteins corresponding to the binding profiles. In many cases one or more binding events produces inconclusive or even aberrant results and this, in turn, can yield ambiguous binding profiles. For example, observation of binding outcomes at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g. positive binding outcomes and/or negative binding outcomes) for one or more affinity reagents with respect to one or more candidate proteins. A binding model can include a measure of the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an extant protein in a sample, an empirical binding profile for the extant protein can be compared to results computed by the binding model for many or all candidate proteins suspected to be in the sample. A machine learning or artificial intelligence algorithm can be used. An algorithm used for decoding can utilize Bayesian inference. In some configurations, identity for an extant protein is determined based on a likelihood of the extant protein being a particular candidate protein given the empirical binding pattern or based on the probability of a particular candidate protein generating the empirical binding pattern. Particularly useful decoding methods are set forth, for example, in U.S. Pat. No. 10,473,654 or U.S. Pat. No. 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. It will be recognized that methods set forth herein that are utilized to decode extant proteins may be useful for other analyte identification assays, provided said analyte identification assays provide a binding profile that can be decoded.

In some configurations of the apparatus and methods set forth herein, one or more proteins can be detected on a solid support. For example, protein(s) can be attached to a solid support, the solid support can be contacted with detection agents (e.g. affinity agents) in solution, the agents can interact with the protein(s), thereby producing a detectable signal, and then the signal can be detected to determine the presence of the protein(s). In multiplexed versions of this approach, different proteins can be attached to different addresses in an array, and the probing and detection steps can occur in parallel. In another example, affinity agents can be attached to a solid support, the support can be contacted with proteins in solution, the proteins can interact with the affinity agents, thereby producing a detectable signal, and then the signal can be detected to determine presence, quantity or characteristics of the proteins. This approach can also be multiplexed by attaching different affinity agents to different addresses of an array.

Proteins, affinity agents or other objects of interest can be attached to a solid support via covalent or non-covalent bonds. For example, a linker can be used to covalently attach a protein or other object of interest to an array. A particularly useful linker is a structured nucleic acid particle such as a nucleic acid nanoball (e.g. a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms a respective address in the array. Exemplary linkers for attaching proteins, or other objects of interest, to an array or other solid support are set forth in U.S. Pat. No. 11,203,612 or U.S. Pat. No. 11,505,796 or US Pat. App. Pub. No. 2023/0167488 A1, each of which is incorporated herein by reference.

In some configurations of the compositions, apparatus and methods set forth herein, one or more proteins can be present on a solid support, where the proteins can optionally be detected. For example, a protein can be attached to a solid support, the solid support can be contacted with a detection agent (e.g. affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses in an array, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

In multiplexed configurations, different proteins can be attached to different unique identifiers (e.g. addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel, allowing for rapid detection of binding events. A plurality of different proteins can have a complexity of at least 5, 10, 100, 1×10³, 1×10⁴, 1×10⁵ or more different native-length protein primary sequences. Alternatively or additionally, a proteome, proteome subfraction or other protein sample that is analyzed in a method set forth herein can have a complexity that is at most 1×10⁵, 1×10⁴, 1×10³, 100, 10, 5 or fewer different native-length protein primary sequences. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A protein can be attached to a unique identifier using any of a variety of means. The attachment can be covalent or non-covalent. Exemplary covalent attachments include chemical linkers such as those achieved using click chemistry or other linkages known in the art or described in U.S. Pat. No. 11,203,612, which is incorporated herein by reference. Non-covalent attachment can be mediated by receptor-ligand interactions (e.g. (strept) avidin-biotin, antibody-antigen, or complementary nucleic acid strands), for example, wherein the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. Pat. Nos. 11,203,612 11,505,796, and US Pat. Pub. No 2023/0167488 A1, each of which is incorporated herein by reference.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event. In particular configurations a flow cell contains the solid support such that fluids introduced to the flow cell can interact with a surface of the solid support to which one or more components of a binding event (or other reaction) is attached.

A method of the present disclosure can be carried out at single analyte resolution. As such, a single analyte (i.e. one and only one analyte), such as a single protein, can be individually manipulated or distinguished using a method set forth herein. A single analyte can be a single molecule (e.g. single protein), a single complex of two or more molecules (e.g. a single protein attached to a structured nucleic acid particle or a single protein attached to an affinity agent), a single particle, or the like. A single analyte may be resolved from other analytes based on, for example, spatial or temporal separation from the other analytes. Reference herein to a ‘single analyte’ in the context of a composition, apparatus or method does not necessarily exclude application of the composition, apparatus or method to multiple single analytes that are manipulated or distinguished individually, unless indicated to the contrary.

Alternatively to single-analyte resolution, a method can be carried out at ensemble-resolution or bulk-resolution. Bulk-resolution configurations acquire a composite signal from a plurality of different analytes or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample. For example, the ensembles can be located at different addresses in an array. Accordingly, the composite signal obtained from each address will be an average of signals from the ensemble yet signals from different addresses can be distinguished from each other.

A particularly useful multiplex format uses an array of analytes (e.g. proteins) and/or affinity agents. The analytes and/or affinity agents can be attached to unique identifiers (e.g. addresses of the array) such that the analytes can be distinguished from each other. An array can be used in any of a variety of processes such as an analytical process used for detecting, identifying, characterizing or quantifying an analyte. Analytes can be attached to unique identifiers via covalent or non-covalent (e.g. ionic bond, hydrogen bond, van der Waals forces etc.) bonds. An array can include different analyte species that are each attached to different unique identifiers. An array can include different unique identifiers that are attached to the same or similar analyte species. An array can include separate solid supports or separate addresses that each bear a different analyte, in which the different analytes can be identified according to the locations of the solid supports or addresses.

An address of an array can contain a single analyte, or it can contain a population of several analytes of the same species (i.e. an ensemble of the analytes). Alternatively, an address can include a population of different analytes. Addresses are typically discrete in an array. Discrete addresses that neighbor each other can be contiguous, or they can be separated by interstitial spaces. An array useful herein can have, for example, addresses that are separated by an average distance of less than 100 microns, 10 microns, 1 micron, 100 nm, 10 nm or less. Alternatively or additionally, an array can have addresses that are separated by an average distance of at least 10 nm, 100 nm, 1 micron, 10 microns, 100 microns or more. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

One or more compositions set forth herein can be present in an apparatus or vessel. For example, a composition of the present disclosure can be present in a vessel, such as a flow cell. As a further option, the vessel can be engaged with a detection apparatus. The vessel can be permanently or temporarily engaged with the detection apparatus. A detection apparatus can be configured to detect the contents of a vessel, for example, by acquiring signals arising from the vessel. For example, a detection apparatus can be configured to acquire optical signals through an optically transparent window of the vessel. Optionally, the detection apparatus can be configured for luminescence detection, for example, having an optical train that delivers radiation from an excitation source (e.g. a laser or lamp) through a window of the vessel. The detection apparatus can further include a camera or other detector that acquires signals transmitted through the window of the vessel and through an optical train. Optionally, excitation and emission can be transmitted through the same optical train; however, separate optical trains can also be useful.

In addition to the foregoing reagents, also provided herein are kits useful in carrying out the analyses described herein, which kits may include the affinity reagents described above. The kits may optionally include one or more of enrichment reagents used to enrich for low abundance proteins and proteoforms, e.g., beads and antibodies used for the immune-isolation and/or immunoprecipitation of the proteins of interest, wash and other elution reagents, for such enrichment. Such kits may also include the flow-cells and arrays used to immobilize proteins of interest in a single molecule, in an optically detectable format for subsequent analysis in appropriately configured optical detection systems described herein. Such kits can include instructions for carrying out the enrichment, flow-cell deposition, interrogation and follow on analysis of biological samples using such kits.

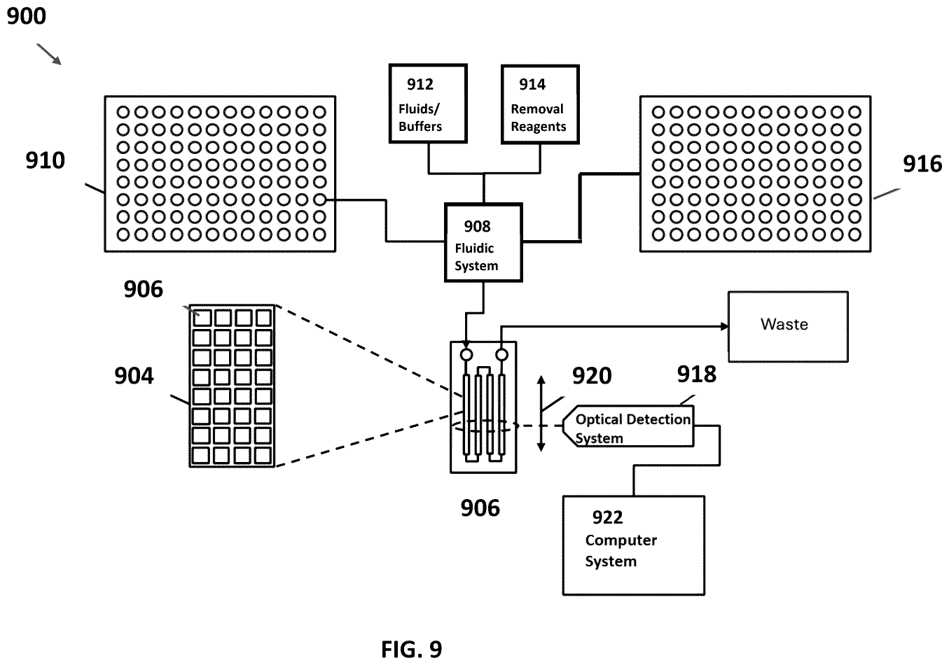

Additionally, provided herein are systems for performing the techniques, reagents, systems, and methods described herein. An example of a system is illustrated in FIG. 9. As shown, the system 900 includes a flowcell 902 that includes an array surface (shown as 904) within the channels of the flow cell upon which individual protein molecules from a sample may be deposited and immobilized in locations 906 that are individually addressable, and in particular cases are individually optically resolvable from each other using, e.g., fluorescence microscopy or scanning techniques.

The system will also typically include a fluidic delivery system 908 that is configured to deliver different fluids to the flow cell 902 through a series of fluidic lines and utilizing appropriate pumps, valves and other conventional fluid controls. The fluidics system 908 may be fluidically coupled to various sources of fluids and reagents needed to carry out the analysis on the flow cell. For example, as shown, fluidic system 908 is fluidly coupled to a source of a plurality of reagents 910 (shown as a 96 well plate, although any number of different reagent storage systems of varying capacity may be employed) that includes a library of multiple affinity reagents that each have affinity for different characteristics of one or more proteins of interest. Additionally, fluidic system 908 may also be coupled to sources of washing fluids or buffers 912, and removal reagents 914 (for removing bound affinity reagents following detection), as well as any other ancillary fluids and reagents needed for the analysis. Similarly, where flow cells are prepared on the system, the fluidic system may be coupled to sources of different sample materials that are to be analyzed 916 (again, shown as a 96 well plate, although again, any suitable sample storage system or capacity may be suitable).

The reagents sources are typically fluidly connected to the flow-cell using fluidics systems that can separately access different reagents, sample materials and other fluids, and control the timing and volume of different reagents delivered to the flow-cell at different times in order to carry out the deposition, interrogation, washing and removal steps of the analysis process. Such fluidic systems will typically include requisite valves and pumps for carrying out such fluid deliveries and include, for example, those as described in, for example, International Patent Application No. WO 2023/122589A2, the full disclosure of which is hereby incorporated herein by reference in its entirety for all purposes.

The systems described herein also typically include a detection system, such as optical detection system 918, for detecting and recording fluorescent signals arising from different positions on the array surface. Such detection systems may generally include line scanning confocal fluorescent microscope systems, which are capable of scanning across large array surfaces (as shown by arrow 920) to detect and record fluorescence across such surfaces at reasonably high scan rates.

The overall systems also typically include one or more computers or processors 922 for controlling the operation of the instrument system including the fluidic system 908 (e.g., to sample different sample sources 916, reagent sources 910 and delivery timing and volume of each), and detection system 918, among other functions, and for recording the detected signals received from the detection system 918, e.g. fluorescent signals, and analyzing such signals to identify potential binding by each of the different affinity reagents. Processors 922 also have access to memory storing instructions that are executed to perform any of the techniques described herein. Included in such memory may be bioinformatic software or firmware that evaluates the signals received and based upon appropriate modeling, identifies likely positive binding events, and then subsequently provides an overall assessment of characteristics of the proteins as described herein including identification information of proteins that are present at any given location on the array and/or the relative abundance of each different protein across the array and ultimately, within the sample being analyzed. Examples of bioinformatic software processes for analyzing such proteoform and proteome data have been described in, for example, U.S. Pat. Nos. 11,545,234, 10,473,654B1, and Egertson, et al., A theoretical framework for proteome-scale single-molecule protein identification using multi-affinity protein binding reagents, bioRxiv, U.S. Patent Application No. 2022/0236282, International Patent Application Nos. PCT/US24/15132, and WO 2023/038859. Alternatively, in some cases, recorded data from the binding events, stored as digital information, digital image files, or compressed versions of such image files, may be transmitted to separate servers or cloud-based systems, which house the informatics software that performs this latter analysis and reporting.

The computer system 922 can be an electronic device of a detection system, the electronic device being integral to the detection system or remotely located with respect to the detection system. The computer system 922 includes a computer processing unit (CPU, also “processor” and “computer processor” herein), which can be a single core or multi-core processor, or a plurality of processors for parallel processing. The computer system 922 also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system 922 can be operatively coupled to a computer network (“network”) with the aid of the communication interface. The network can be the Internet, an internet and/or extranet, or an intranet and/or extranet that is in communication with the Internet. The network, in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, receiving information of empirical measurements of analytes in a sample; processing information of empirical measurements against a database comprising a plurality of candidate analytes, for example, using a binding model or function set forth herein; generating probabilities of a candidate analytes generating empirical measurements, and/or generating probabilities that extant analytes are correctly identified in the sample, and/or determining abundances of analytes in the sample. Such cloud computing may be provided by cloud computing platforms such as, for example, Amazon Web Services (AWS), Microsoft Azure, Google Cloud Platform, and IBM cloud. The network, in some cases with the aid of the computer system 922, can implement a peer-to-peer network, which may enable devices coupled to the computer system 922 to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system 922 can be included in the circuit. In some cases, the circuit is an application specific integrated circuit (ASIC).

The storage unit can store files, such as drivers, libraries and saved programs. The storage unit can store user data, e.g., user preferences and user programs. The computer system 922 in some cases, can include one or more additional data storage units that are external to the computer system 922, such as located on a remote server that is in communication with the computer system 922 through an intranet or the Internet.

The computer system 922 can communicate with one or more remote computer systems through the network. For instance, the computer system 922 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 922 via the network.

Methods as described herein can be implemented by way of machine (e.g., computer processor) executable code stored on an electronic storage location of the computer system 922, such as, for example, on the memory or electronic storage unit. The machine executable or machine readable code can be provided in the form of software. During use, the code can be executed by the processor. In some cases, the code can be retrieved from the storage unit and stored on memory for ready access by the processor. In some situations, the electronic storage unit can be precluded, and machine-executable instructions are stored on memory.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 922, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, may also be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system 922 can include or be in communication with an electronic display that comprises a user interface (UI) for providing, for example, user selection of algorithms, binding measurement data, candidate proteins, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, receive information of empirical measurements of extant proteins in a sample, compare information of empirical measurements against a database comprising a plurality of protein sequences corresponding to candidate proteins, generate probabilities of a candidate protein generating the observed measurement outcome profile, and/or generate probabilities that candidate proteins are correctly identified in the sample, and/or generate abundances for the proteins in the sample.

The present disclosure provides a non-transitory information-recording medium that has, encoded thereon, instructions for the execution of one or more steps of the methods or techniques set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

A solid support or a surface thereof may be configured to display an analyte or a plurality of analytes. A solid support may contain one or more addresses in formed or prepared surfaces. Multiple addresses can be configured to form a pattern. In some cases, a solid support may contain one or more patterned, formed, or prepared surfaces that contain a plurality of addresses, with each address configured to display one or more analytes. A solid support or a surface thereof may contain one or more structures or features. A structure or feature may comprise an elevation, profile, shape, geometry, or configuration that deviates from an average elevation, profile, shape, geometry, or configuration of a solid support or surface thereof. A structure or feature may be a raised structure or feature, such as a ridge, post, pillar, or pad, if the structure or feature extends above the average elevation of a surface of a solid support. A structure or feature may be a depressed structure, such as a channel, well, pore, or hole, if the structure or feature extends below the average elevation of a surface of a solid support. A solid support or a surface thereof may be formed by a suitable lithographic method, including, but not limited to photolithography, Dip-Pen nanolithography, nanoimprint lithography, nanosphere lithography, nanoball lithography, nanopillar arrays, nanowire lithography, immersion lithography, neutral particle lithography, plasmonic lithography, scanning probe lithography, thermochemical lithography, thermal scanning probe lithography, local oxidation nanolithography, molecular self-assembly, stencil lithography, laser interference lithography, soft lithography, magnetolithography, stereolithography, deep ultraviolet lithography, x-ray lithography, ion projection lithography, proton-beam lithography, or electron-beam lithography.

A solid support or surface may comprise a plurality of structures or features. Structures or features may be provided as analyte-binding sites for the coupling of analytes or other moieties (e.g., anchoring moieties). A plurality of structures or features may comprise a repeating pattern of structures or features. A plurality of structures or features may comprise a non-ordered, non-repeating, or random distribution of structures or features. A structure or feature may have an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature may have an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. An array of structures or features may have an average pitch, in which the pitch is measured as the average separation between respective centerpoints of adjacent structures or features. An array may have an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array may have an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

A method of the present disclosure can include the step of coupling one or more analytes to a solid support or a surface thereof, for example, prior to performing a detection step set forth herein. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

An array of analytes may be provided for a method, composition, system, or apparatus set forth in the present disclosure. Although analytes are exemplified as proteins throughout the present disclosure, it will be understood that other analytes may be provided in a similar array format. Exemplary analytes include, but are not limited to, cells, organelles, biomolecules, polysaccharides, nucleic acids, lipids, metabolites, hormones, vitamins, enzyme cofactors, therapeutic agents, candidate therapeutic agents, or combinations thereof. An analyte can be a non-biological atom or molecule, such as a synthetic polymer, metal, metal oxide, ceramic, semiconductor, mineral, or a combination thereof.

Array analyte-binding sites can comprise one or more moieties that are coupled or otherwise bound to a solid support at the analyte-binding site. Moieties may be bound to a solid support at an analyte-binding site for facilitating coupling of an analyte to the analyte-binding site, or to inhibit unwanted binding of moieties to the analyte-binding site. Moieties may be covalently or non-covalently bound to a solid support at an analyte-binding site.

An analyte-binding site may be provided with one or more moieties that couple an analyte to the analyte-binding site. Coupling moieties can include non-covalent coupling moieties (e.g., oligonucleotides, receptor-ligand binding pairs, electrically-charged moieties, magnetic moieties, etc.), or covalent coupling moieties (e.g., Click-type reactive groups, etc.). An analyte-binding site may be provided with one or more passivating moieties that inhibit unwanted or unexpected binding of moieties to the analyte-binding site. Exemplary passivating moieties can include polymeric molecules such as polyethylene glycol (PEG), bovine serum albumin, pluronic F-127, polyvinylpyrrolidone, and Teflon, or hydrophobic materials such as hexamethyldisilazane. A passivating moiety may be covalently or non-covalently bound to a solid support at an analyte-binding site. An analyte-binding site may contain a covalently bound passivating moiety and a non-covalently bound passivating moiety. For example, an analyte-binding site may contain a PEG moiety that is covalently attached to the solid support at the analyte-binding site and a bovine serum albumin moiety that is electrostatically bound to the analyte-binding site.

Analytes may be attached directly to analyte-binding sites, for example, by coupling of a moiety attached to an analyte to a moiety attached to an analyte-binding site. Alternatively, analytes may be attached to analyte-binding sites by an anchoring moiety. An anchoring moiety may attach an analyte to an analyte-binding site, and optionally orient the analyte and/or occlude additional analytes from attaching to the analyte-binding site. An anchoring moiety may comprise a nanoparticle, such as a metal nanoparticle, a metal oxide nanoparticle, a semiconductor nanoparticle, a carbon nanoparticle, or a polymeric nanoparticle. Preferably, an anchoring moiety may comprise a nucleic acid nanoparticle. A nucleic acid nanoparticle of an anchoring moiety may comprise a first face containing one or more coupling moieties, and a second face containing an analyte-coupling site. The first face and the second face of the anchoring moiety may be substantially opposed. The anchoring moiety may further comprise a linking moiety that attaches the analyte to the anchoring moiety. The linking moiety may spatially separate the analyte from the surface of the array, for example by a distance of at least about 5 nm, 10 nm, 15 nm, 20 nm, 25 nm, 30 nm, 40 nm, 50 nm, or more than 50 nm. The linking moiety may comprise a flexible linker (e.g., a PEG or alkyl moiety) or a rigid linker (e.g., a double-stranded nucleic acid linker). An anchoring moiety may be attached to one and only one analyte. An anchoring moiety may be attached to more than one analyte. Additional aspects of anchoring moieties are described in U.S. Pat. Nos. 11,203,612, and 11,505,796, and U.S. Pat. Pub. No. 20240417720A1, each of which is incorporated herein by reference in its entirety.

An anchoring moiety may be provided with moieties that facilitate a binding interaction with a surface of a solid support, or moieties coupled to the surface of the solid support. Moieties that facilitate coupling of an anchoring moiety to a solid support may be configured to form a covalent interaction or a non-covalent interaction with the solid support or a moiety coupled to the solid support. In an example, an anchoring moiety may be provided with one or more nucleic acid strands that can hybridize to a complementary nucleic acid strand on a surface of a solid support by nucleic acid hybridization. Preferably, an anchoring moiety may be provided with a plurality of moieties that can bind to a surface of a solid support. In some cases, the moieties may be pendant from the anchoring moiety. Pendant moieties may include a linking moiety that increases the length of the moiety and/or increases the flexibility or spatial degrees of freedom of the moiety. A linking moiety can be, for example, a single-stranded nucleic acid (e.g., with a nucleotide sequence that is not complementary to a surface-bound oligonucleotide), a peptide linker, or a synthetic polymer (e.g., polyethylene glycol, alkyl moieties, etc.).

It may be especially useful to provide an array of analytes with a diversity of polypeptide species. The diversity of polypeptide species may be measured with respect to a proteome, sub-proteome (e.g., a tissue proteome, a cell proteome, an organelle proteome, a metabolome, a signalome, an albuminome, etc.), or a microbiome. An array of analytes may be provided with a diversity of polypeptide species as measured by total number of polypeptide species, percentage of species of a proteome, subproteome, or microbiome, number of proteoforms of a polypeptide species, or polypeptide dynamic range.

An array of analytes may be provided with more than one unique species of polypeptide. A first polypeptide may be considered unique from a second polypeptide if the amino acid sequences of the first polypeptide and second polypeptide differ. An array of analytes may be provided with at least about 2, 5, 10, 50, 100, 500, 1000, 2000, 5000, 10000, 15000, 20000, 25000, 30000, 40000, 500000, 100000, or more than 100000 unique species of polypeptides. Alternatively or additionally, an array of analytes may be provided with no more than about 100000, 50000, 40000, 30000, 25000, 20000, 15000, 10000, 5000, 2000, 1000, 500, 100, 50, 10, 5, 2, or less than 2 unique species of polypeptides.

An array of analytes may be provided with a fraction or percentage of species of a proteome, subproteome, or microbiome. An array of analytes may be provided with at least about 0.1%, 1%, 5%, 10%, 15%, 20%, 25%, 30%, 35%, 40%, 45%, 50%, 55%, 60%, 65%, 70%, 75%, 80%, 85%, 90%, 95%, 99%, 99.9%, or more than 99.9% of polypeptide species of a proteome, subproteome, or microbiome. Alternatively or additionally, an array of analytes may be provided with no more than about 99.9%, 99%, 95%, 90%, 85%, 80%, 75%, 70%, 65%, 60%, 55%, 50%, 45%, 40%, 35%, 30%, 25%, 20%, 15%, 10%, 5%, 1%, 0.1%, or less than 0.1% of polypeptide species of a proteome, subprotcome, or microbiome.

An array of analytes may be provided with more than one proteoform of a polypeptide species. An array of analytes may be provided with more than one proteoform for two or more unique polypeptide species. Types of proteoforms of a polypeptide species can include coding variation proteoforms, translational variation proteoforms, post-translational modification proteoforms, splice variants, and combinations thereof. An array of analytes may be provided with at least about 2, 3, 4, 5, 10, 20, 50, 100, 200, 500, 1000, or more than 1000 proteoforms of a polypeptide species. Alternatively or additionally, an array of analytes may be provided with no more than about 1000, 500, 200, 100, 50, 20, 10, 5, 4, 3, or less than 3 proteoforms of a polypeptide species.

An array of analytes may be provided with a dynamic range of polypeptides. Dynamic range can refer to the ratio of abundance between a more populous polypeptide species and a less populous polypeptide species. A dynamic range can be an absolute measure (ratio of most populous polypeptide species to least populous polypeptide species) or a relative measure (ratio of a first particular polypeptide species to a second particular polypeptide species). An array of analytes may be provided with a dynamic range of at least about 10, 10², 10³, 10⁴, 10⁵, 10⁶, 10⁷, 10⁸, 10⁹, 10¹⁰, 10¹¹, 10¹², or more than 10¹². Alternatively or additionally, an array of analytes may be provided with a dynamic range of no more than about 10¹², 10¹¹, 10¹⁰, 10⁹, 10⁸, 10⁷, 10⁶, 10⁵, 10⁴, 10², 10², 10, or less than 10.

In some methods, providing an array of analytes may further comprise forming the array of analytes. An array of analytes may be formed by a process that includes a step of coupling analytes to analyte-binding sites of the array. An analyte may be coupled to an analyte-binding site by coupling of a coupling moiety attached to the analyte to a compatible coupling moiety attached to the analyte-binding site. In some cases where an analyte is attached to an anchoring moiety, a step of coupling the analyte to the analyte-binding site may comprise coupling the anchoring moiety to the analyte-binding site. In particular cases, an analyte may be coupled to an analyte-binding site by coupling of a coupling moiety attached to an anchoring moiety to a compatible coupling moiety attached to the analyte-binding site. A fluidic medium containing a plurality of analytes may be contacted to a solid support comprising a plurality of analyte-binding sites. After contacting the fluidic medium comprising the analytes to the solid support, analytes may couple to analyte-binding sites, thereby forming the array of analytes.

Entities, such as affinity reagents and their binding targets, can be associated with each other and dissociated form each other in a method set forth herein. Association of a first entity to a second entity can involve a contacting step, in which the first entity is brought into proximity of the second entity, and an association step in which a first coupling moiety of the first entity forms a binding interaction with a second coupling moiety of the second entity. Dissociation of a first entity and a second entity need not be construed as a reversal of an association process between the first entity and the second entity. For example, a first entity comprising a first oligonucleotide coupled to a second entity comprising a second oligonucleotide by hybridization of the first oligonucleotide to the second oligonucleotide could be dissociated by dehybridization of the nucleic acids (thereby returning the first entity and the second entity as originally provided before association), or dissociated by enzymatic cleavage of the hybridized nucleic acids (thereby providing the first and the second entities with each individually further comprising an at least partially double-stranded cleavage product).

Systems or methods set forth herein may utilize one or more fluidic media to implement a process or step thereof. For array-based processes and systems, fluidic media may be provided for various process steps, including preparing arrays, attaching analytes to arrays, associating affinity agents to analytes, dissociating affinity agents from analytes, rinsing unbound moieties from array surfaces, performing detection processes on arrays, displacing a fluidic medium from contact with an array or other system components, and various other chemical and/or physical alterations of analytes or array components. A fluidic medium may be formulated to deliver a plurality of macromolecules (e.g., analytes, affinity agents) to an array as set forth herein. A fluidic medium may be formulated to mediate an interaction between macromolecules (e.g., an interaction between an analyte and an affinity agent).

A fluidic medium may be a single-phase or multi-phase fluidic medium. A multi-phase fluidic medium can include a gas phase and a liquid phase or at least two immiscible liquids. A multi-phase fluidic medium may comprise an interface between a first phase and a second phase. An interface between two fluidic phases may be laminar (e.g., an oil phase floating on an aqueous phase) or dispersed (e.g., bubbles, vesicles or droplets). A dispersed interface may be formed by a process such as emulsification. A divided interface may be stable (e.g., an emulsion) or unstable (e.g., a flocculating suspension). A multi-phase fluidic medium may comprise a colloidal agent that mediates an interface between a first phase and a second phase.

A fluidic medium can further contain solids, including particles (e.g., microparticles, nanoparticles). A fluidic medium comprising solids may be provided as a mixture, a suspension, or a slurry. It may be advantageous to provide a fluidic medium comprising a mixture or suspension of macromolecules. In some cases, solubility or suspendability of solids, such as particles or macromolecules, within a fluidic medium can be modulated by the composition of the fluidic medium. For example, alteration of fluidic properties such as solvent composition, ionic strength, and/or pH can induce precipitation, sedimentation, or flocculation of solvated or suspended solids.

A fluidic medium may be formulated with any one of numerous components depending upon its intended application. A fluidic medium can comprise one or more solvents. A single-phase fluidic medium can comprise two or more miscible solvents. In a mixture of miscible solvents, a solvent may be considered a base solvent if it comprises a greater than 50% fraction on a mass, molar, or volumetric basis. A miscible solvent may be mixed into a base solvent to alter a physical property of the base solvent, such as polarity, density, pH, viscosity, or surface tension. A fluidic medium can comprise a polar solvent or a non-polar solvent. A fluidic medium can comprise a protic or aprotic solvent. A fluidic medium can comprise an aqueous medium. A fluidic medium can comprise an organic solvent, such as acetic acid, acetone, acetonitrile, benzene, a butanol, 2-butanone, carbon tetrachloride, chlorobenzene, chloroform, cyclohexane, 1,2-dichloroethane, diethylene glycol, diethyl ether, diglyme, 1,2-dimethoxy-ethane, dimethylformamide, dimethyl sulfoxide, 1,4-dioxane, ethanol, ethyl acetate, ethylene glycol, glycerin, heptane, hexamethylphosphoramide, hexamethylphophorus triamide, hexanes, methanol, methyl t-butyl ether, methylene chloride, N-methyl-pyrrolidinone, nitromethane, pentane, petroleum ether, 1-proponal, 2-propanol, pyridine, tetrahydrofuran, toluene, triethyl amine, xylene, or a combination thereof. A fluidic medium can comprise a polar solvent, such as N-methyl pyrrolidone, tetrahydrofuran, ethyl acetate, acetone, dimethylfuran, acetonitrile, dimethyl sulfoxide, propylene carbonate, N-butanol, isopropyl alcohol, nitromethane, ethanol, methanol, acetic acid, or a combination thereof. A fluidic medium can comprise a non-polar solvent, such as benzene, carbon tetrachloride, chloroform, cyclohexane, dichloromethane, dimethoxyethane, ethyl ether, heptane, hexachloroethane, hexane, limonene, naphtha, pentane, tetrachloroethylene, tetrahydrofuran, toluene, xylenes, and combinations thereof. In some cases, a fluidic medium may comprise an aprotic solvent, such as N-methyl pyrrolidone, tetrahydrofuran, ethyl acetate, acetone, dimethylfuran, acetonitrile, dimethyl sulfoxide, propylene carbonate, or a combination thereof.

A fluidic medium may further comprise one or more components, including: 1) an ionic species, 2) a buffering agent, 3) a surfactant or detergent, 4) a chelating agent, 5) a denaturing agent or a chaotrope, 6) a cosmotropic or crowding agent, 7) a clouding agent, 8) a reactive scavenger, and 9) a blocking agent.

A fluidic medium may comprise one or more ionic species. An ionic species may be provided to a fluidic medium as a salt, thereby providing an anionic species and a cationic species to the fluidic medium. An ionic species can include a zwitterionic species. A fluidic medium may comprise a cationic species such as Na⁺, K⁺, Ag⁺, Cu⁺, NH⁴⁺, Mg²⁺, Ca²⁺, Cu²⁺, Cd²⁺, Zn²⁺, Fc²⁺, Co²⁺, Ni²⁺, Cr²⁺, Mn²⁺, Ge²⁺, Sn²⁺, Al³⁺, Cr³⁺, Fe³⁺, Co³⁺, Ni³⁺, Ti³⁺, Mn³⁺, Si⁴⁺, V⁴⁺, Ti⁴⁺, Mn⁴⁺, Ge⁴⁺, Sc⁴⁺, V⁵⁺, Mn⁵⁺, Mn⁶⁺, Se⁶⁺, and combinations thereof. A fluidic medium may comprise an anionic species such as F⁺, Cl⁻, Br⁻, ClO₃⁻, H₂PO₄⁻, HCO₃⁻, HSO₄⁻, OH⁻, I⁻, NO₃⁻, NO₂⁻, MnO₄⁻, SCN⁻, CO₃²⁻, CrO₄²⁻, Cr₂O₇²⁻, HPO₄²⁻, SO₄²⁻, SO₃²⁻, PO₄³⁻, and combinations thereof. A fluidic medium may comprise a chelating agent, such as ethylenediaminetetraacetic acid (EDTA), nitrilotriacetic acid, n-hydroxyethylenediaminetetraacetic acid (HEDTA), oxalic acid, malic, acid, rubeanic acid, citric acid, or combinations thereof.

A fluidic medium may include a buffering species including, but not limited to, MES, Tris, Bis-tris, Bis-tris propane, ADA, ACES, PIPES, MOPSO, MOPS, BES, TES, HEPES, HEPBS, HEPPSO, DIPSO, MOBS, TAPSO, TAPS, TABS, POPSO, TEA, EPPS, Tricine, Gly-Gly, Bicine, AMPD, AMPSO, AMP, CHES, CAPSO, CAPS, PBS, and CABS.

A fluidic medium may comprise a surfactant or detergent. A surfactant or detergent may comprise a cationic surfactant or detergent, an anionic surfactant or detergent, a zwitterionic surfactant or detergent, an amphoteric surfactant or detergent, or a non-ionic surfactant or detergent. A fluidic medium may include a surfactant species including, but not limited to, stearic acid, lauric acid, oleic acid, sodium dodecyl sulfate, sodium dodecyl benzene sulfonate, dodecylamine hydrochloride, hexadecyltrimethylammonium bromide, polyethylene oxide, nonylphenyl ethoxylates, Triton X, pentapropylene glycol monododecyl ether, octapropylene glycol monododecyl ether, pentacthylene glycol monododecyl ether, octaethylene glycol monododecyl ether, lauramide monoethylamine, lauramide diethylamine, octyl glucoside, decyl glucoside, lauryl glucoside, Tween 20, Tween 80, n-dodecyl-β-D-maltoside, nonoxynol 9, glycerol monolaurate, polyethoxylated tallow amine, poloxamer, digitonin, zonyl FSO, 2,5-dimethyl-3-hexyne-2,5-diol, Igepal CA630, Aerosol-OT, triethylamine hydrochloride, cetrimonium bromide, benzethonium chloride, octenidine dihydrochloride, cetylpyridinium chloride, adogen, dimethyldioctadecylammonium chloride, CHAPS, CHAPSO, cocamidopropyl betaine, amidosulfobetaine-16, lauryl-N,N-(dimethylammonio) butyrate, lauryl-N,N-(dimethyl)-glycinebetaine, hexadecyl phosphocholine, lauryldimethylamine N-oxide, lauryl-N,N-(dimethyl)-propanesulfonate, 3-(1-pyridinio)-1-propanesulfonate, 3-(4-tert-butyl-1-pyridinio)-1-propanesulfonate, N-laurylsarcosine, and combinations thereof.

A fluidic medium may comprise a denaturing or chaotropic species, such as acetic acid, trichloroacetic acid, sulfosalicylic acid, sodium bicarbonate, ethanol, ethylenediamine tetraacetic acid (EDTA), urea, guanidinium chloride, lithium perchlorate, sodium dodecyl sulfate, 2-mercaptoethanol, dithiothreitol, tris(2-carboxyethyl) phosphine (TCEP), or a combination thereof. A denaturing or chaotropic species may be provided to alter a conformational state of an array component (e.g., causing denaturation of a polypeptide), or may be provided to maintain a conformational state of an array component (e.g., maintaining a polypeptide in a denatured or partially-denatured state).

A fluidic medium may comprise a cosmotropic species, such as carbonate ion, sulfate ion, phosphate ion, magnesium ion, lithium ion, zinc ion, aluminum ion, trehalose, glucose, proline, tert-butanol, or a combination thereof. A fluidic medium may comprise a clouding agent such as sodium chloride, potassium chloride, sodium bromide, potassium bromide, sodium nitrate, sodium sulfate, sodium phosphate, or a combination thereof. A cosmotropic species may be provided to decrease a separation distance between molecules and array components (e.g., causing smaller separation between an affinity agent and an analyte).

A fluidic medium may comprise a reactive scavenger species. A reactive scavenger may be provided to reduce solution-phase concentrations of reactive species (e.g., oxidizing or reducing species). A reactive scavenger may be provided during a photon-mediated process (e.g., fluorescent imaging) to reduce photodamage or other deleterious photon-related processes (e.g., singlet oxygen generation, free radical generation). Exemplary reactive scavenger species can include ascorbic acid, 9,10-anthracenediyl-bis(methylene)dimalonic acid (ABDA), epigallocatechin gallate (EPGG), N-acetyl-L-cysteine, caffeic acid, reseveratrol, 4-hydroxy-2,2,6,6-tetramethylpiperidin-1-oxyl (TEMPOL), sodium sulfite, 1,4-diazabicyclo[2.2.2]octane (DABCO), sodium pyruvate, N,N′-dimethylthiourea (DMTU), mannitol, dimethyl sulfoxide (DMSO), 6-hydroxy-2,5,7,8-tetramethylchroman-2-carboxylic acid (Trolox), 2-phenyl-1,2-benzisoselenazol-3(2H)-one (Ebselen), α-tocopherol, uric acid, sodium azide, manganese (III)-tetrakis (4-benzoic acid) porphyrin, 4,5-dihydroxybenzene-1,3-disulfonate, or a combination thereof. Other useful reactive scavengers and methods for their use in reducing photodamage or other deleterious photon-related processes are set forth in U.S. Pat. No. 10,106,851, which is incorporated herein by reference.

A fluidic medium may comprise a blocking agent. A blocking agent may include any species that inhibits orthogonal binding phenomena between assay agents and array components, such as polyethylene glycol, dextrans, albumin, or synthetic polymers such as PF-127 or polyvinylpyrrolidone.

A method set forth herein may involve a step of delivering a fluidic medium to a vessel (e.g., a flow cell, a fluidic cartridge, a reactor or microreactor, etc.) containing an array, as set forth herein. In some cases, after delivering a fluidic medium to a vessel, the fluidic medium may be incubated with an array within the vessel. Incubation of a fluidic medium with an array may be substantially quiescent. Alternatively, incubation of a fluidic medium with an array may be non-quiescent due to mixing, agitation, or circulation of the fluidic medium within or through the vessel.

The methods, compositions and apparatus of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof.

One or more proteins that are used in a method, composition or apparatus herein, can be derived from a natural or synthetic source. Exemplary sources include, but are not limited to biological tissues, fluids, cells or subcellular compartments (e.g. organelles). For example, a sample can be derived from a tissue biopsy, biological fluid (e.g. blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharomyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell or biological fluid that is known or suspected of being linked with a disease state or disorder (e.g., cancer). Alternatively, a protein can be derived from an organism, tissue, cell or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g. an individual human), single tissue, single cell, single organelle (e.g. endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 μg and 500 pg depending upon cells type. Sec Wisniewski et al. Molecular & Cellular Proteomics 13:10.1074/mcp.M113.037309, 3497-3506 (2014), which is incorporated herein by reference. A plurality of proteins used or included in a method, composition or apparatus set forth herein can include at least 1 μg, 10 pg, 100 μg, 1 ng, 10 ng, 100 ng, 1 mg, 10 mg, 100 mg, 1 mg, 10 mg, 100 mg or more protein by mass. Alternatively or additionally, a plurality of proteins may contain at most 100 mg, 10 mg, 1 mg, 100 mg, 10 mg, 1 mg, 100 ng, 10 ng, 1 ng, 100 μg, 10 pg, 1 μg or less protein by mass.

A plurality of proteins can be characterized in terms of percent mass relative to a given source such as a biological source (e.g. cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. Sec Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×1023 molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

In relative terms, a plurality of proteins used or included in a method, composition or apparatus set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants ac included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different primary protein structures. Alternatively or additionally, a plurality of proteins can have a complexity that is at most 1×10⁶, 1×10⁵, 7×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Protoeforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

The present disclosure provides compositions, apparatus and methods that are useful for detecting, characterizing and identifying proteoforms. For example, the presence or absence of a particular post-translational modification or a particular post-translationally modified amino acid can be determined. In some embodiments, a proteoform can be characterized with respect to the location(s) of one or more post-translational modifications in the amino acid sequence of the proteoform. Locations can be identified, for example, at a specific position of the amino acid sequence for the proteoform. However, in some cases, the location of a post-translational modification in a proteoform can be determined relative to a particular structural motif of the proteoform. For example, a post-translational moiety of a proteoform can be located relative to a short sequence of amino acids in the proteoform or relative to another post-translational moiety in the proteoform.

Methods of the present disclosure are particularly well suited for manipulating and detecting proteoforms. The presence or absence of post-translational modifications (PTM) can be detected using a composition, apparatus or method set forth herein. A PTM can be detected using an affinity agent that recognizes the PTM or based on a chemical property of the PTM. In some configurations, methods set forth herein can be used to differentially manipulate proteoforms based on unique molecular properties or to distinguish one proteoform from another.

A post-translational modification may be one or more of myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization. Proteoforms can differ with regard to the presence or absence of a post-translational modification, type of post-translational modification present, location of a post-translational modification, number of post-translational modifications present or combination thereof.

A post-translational modification may occur at a particular type of amino acid residue in a protein. For example, the phosphate moiety of a particular proteoform can be present on a serine, threonine, tyrosine, histidine, cysteine, lysine, aspartate or glutamate residue. In another example, an acetyl moiety of a particular proteoform can be present on the N-terminus or on a lysine of a protein. In another example, a serine or threonine residue of a proteoform can have an O-linked glycosyl moiety, or an asparagine residue of a proteoform can have an N-linked glycosyl moiety. In another example, a proline, lysine, asparagine, aspartate or histidine amino acid of a proteoform can be hydroxylated. In another example, a proteoform can be methylated at an arginine or lysine amino acid. In another example, a proteoform can be ubiquitinated at the N-terminal methionine or at a lysine amino acid.

A post-translationally modified version of a given amino acid can include a post-translational moiety at a side chain position that is unmodified in a standard version of the amino acid. Post-translationally modified lysines can include epsilon amines attached to post-translational moieties, whereas standard lysines have epsilon amines lacking the post-translational moieties. Post-translationally modified histidines can include side-chain tertiary amines attached to post-translational moieties, whereas in standard histidines the side-chain amines are secondary amines lacking the post-translational moieties. Post-translationally modified versions of aspartates or glutamates can include side-chain carbonyls, esters or amides attached to post-translational moieties, whereas in standard versions of aspartates or glutamates the side-chains have carboxyls lacking the post-translational moieties. Post-translationally modified versions of arginines can include side-chain amines attached to post-translational moieties, whereas in standard versions of arginines the side-chain amines lack the post-translational moieties. Post-translationally modified versions of cysteines can include thioethers attached to post-translational moieties, whereas standard versions of cysteines have sulfurs lacking the post-translational moieties. Post-translationally modified versions of serines, threonines or tyrosines can include ethers or esters attached to post-translational moieties, whereas standard versions of serines, threonines or tyrosines have hydroxyls lacking the post-translational moieties.

A method of the present disclosure can include a step of removing post-translational moieties from post-translationally modified amino acids, thereby forming standard amino acids. In some cases, an enzyme can be used to remove a post-translational moiety from an amino acid. An enzyme that removes a post-translational moiety independently of amino acid sequence context surrounding the post-translationally modified amino acid can be used. In other cases, a sequence-specific enzyme can be used to remove a post-translational moiety.

A method of characterizing extant proteins may further include identifying proteoforms of extant proteins. Accordingly, a method of identifying a proteoform of an individual protein can include the steps of: i) identifying a primary amino acid sequence of the protein based upon a binding profile of the protein, thereby identifying the protein, and ii) identifying a proteoform of the protein. Proteoform-specific affinity agents may be useful for identifying the proteoform of an extant protein. A proteoform-specific affinity agent can be a promiscuous affinity agent, for example binding to post-translational modifications (e.g., methylations, phosphorylations, glycosylations, etc.) of a plurality of protein species and/or proteoforms. A proteoform-specific affinity agent can be highly specific to a single proteoform of one or more protein species (e.g., only binding to a single post-translationally modified amino acid of a single protein species). A proteoform may be identified in part by detecting the presence of binding of one or more affinity agents to an extant protein. Alternatively, a proteoform may be identified in part by an absence of detectable binding of one or more affinity agents to an extant protein (e.g., due to absence of a post-translational modification at an amino acid residue of the extant protein, due to absence of a bindable epitope due to splice variation of the extant protein, etc.).

In some cases, it may be preferable to contact extant proteins with a proteoform-specific affinity agent before contacting the extant proteins with other promiscuous or non-proteoform affinity agents. The presence of certain post-translational modification may inhibit binding of affinity agents to epitopes where said post-translational modification are present. Accordingly, a method may further comprise a step of removing post-translation modification (e.g., chemically or enzymatically) from extant proteins. After detecting binding of proteoform-specific affinity agents to extant proteins, and optionally removing one or more post-translational modification from the extant proteins, the extant proteins may be subsequently contacted with a series of promiscuous affinity agents, thereby providing binding profiles for each individual extant protein.

### EXAMPLES

### Example 1. Characterization of Molecules by Dissociation Conditions

An array is provided containing a plurality of immobilized protein molecules. Each individual protein molecule of the plurality of immobilized protein molecules is attached to a different array address. Protein molecules are each individually attached to a single nucleic acid nanoparticle that is bound to the solid support of the array. Each protein molecule of the plurality of protein molecules contains one or more phosphorylation post-translational modifications. Binding reagents with a binding specificity for phosphorylated post-translation modifications are coupled to molecules of the plurality of immobilized protein molecules. Each binding reagent is labeled with fluorescent labels that fluoresce at 680 nm. The presence or absence of a 680 nm signal is detected at each address of the array to determine the presence or absence of a binding reagent at each array address.

A first binding reagent dissociation medium containing 100 millimolar (mM) CHAPS surfactant in water is contacted to the array containing the bound binding reagents. After 10 minutes of incubation with the first binding reagent dissociation medium, any dissociated binding reagents are rinsed from the array. After rinsing, the array is imaged, thereby determining the presence or absence of a binding reagent at each array address. By comparison between detection before and after providing the first binding reagent dissociation medium, sites containing a presence of a signal before, and an absence of a signal after, are identified and quantitated against total sites having a presence of the signal before providing the first dissociation medium. In the presence of the first binding reagent dissociation medium, less than 20% of binding reagents are observed to dissociate from phosphorylated protein molecules.

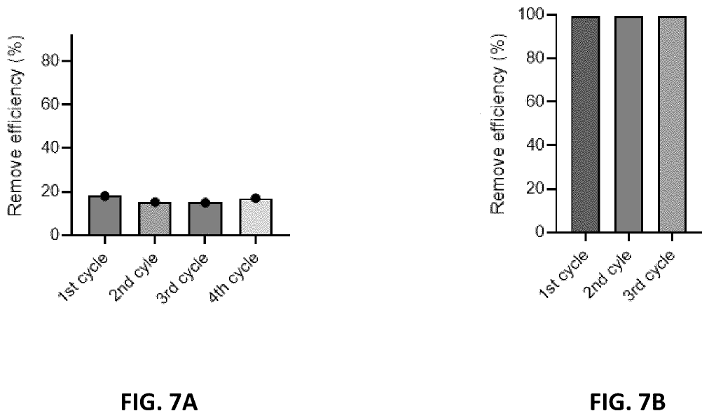

After the post-rinsing detection step, additional binding reagents are contacted to the array. Another cycle of detection, 10 min incubation with the first binding reagent dissociation medium, and post-rinsing detection is performed. The cycle is repeated until a total of four cycles of detection have been performed. FIG. 7A depicts a bar chart of binding reagent dissociation for each cycle, showing consistently less than 20% dissociation of binding reagents in the presence of the first binding reagent dissociation medium.

Binding reagents are again bound to protein molecules of the array. After detecting addresses containing bound binding reagents, a second binding reagent dissociation medium containing 1 wt % sodium dodecyl sulfate, 20 mM sodium acetate, and 20 mM magnesium chloride adjusted to pH 4.25 is contacted to the array containing the bound binding reagents. After 10 minutes of incubation with the second binding reagent dissociation medium, dissociated binding reagents are rinsed from the array. After rinsing, the array is imaged, thereby determining the presence or absence of a binding reagent at each array address. By comparison between detection before and after providing the second binding reagent dissociation medium, sites containing a presence of a signal before and an absence of a signal after are identified and quantitated against total sites having a presence of the signal before providing the second dissociation medium. In the presence of the second binding reagent dissociation medium, 97% of binding reagents are observed to dissociate from phosphorylated protein molecules. Two additional cycles of before and after detection are performed. FIG. 7B depicts a bar chart of binding reagent dissociation for each cycle, showing consistently 97% dissociation of binding reagents in the presence of the second binding reagent dissociation medium. The dissociation specificity of the binding reagent for the phosphorylated protein molecules is now characterized for the two binding reagent dissociation conditions.

### Example 2. Dissociation of Binding Reagents from Protein Targets

Arrays of proteins were prepared. Two arrays comprised transferrin molecules bound to silicon substrates. Two other arrays comprised Q11MAO molecules bound to silicon substrates. Each array was contacted with a sequence of binding reagents. Each cycle comprised: 1) incubation of a plurality of binding reagents with an array; 2) rinsing of unbound binding reagents from the array; 3) detection of fluorescent signals from binding reagents bound to protein molecules of the array; 4) incubation of the array with a binding reagent dissociation medium for 10 minutes; and 5) detection of fluorescent signals from binding reagents bound to protein molecules of the array after contacting with the binding reagent dissociation medium.

Binding reagents were contacted against protein molecules, each had an individually characterized affinity for a particular trimer amino acid target. Tested trimer targets are listed in Table III.

TABLE III

| Cycle | Target |
|---|---|
| 1 | None |
| 2 | None |
| 3 | None |
| 4 | YFS |
| 5 | YF |
| 6 | HIE |
| 7 | AYI |
| 8 | DPY |
| 9 | WNK |
| 10 | NRV |
| 11 | LEF |
| 12 | LSH |
| 13 | LPQ |
| 14 | SLW |
| 15 | CANT |
| 16 | DDY |
| 17 | IRA |
| 18 | SIF |
| 19 | RDE |
| 20 | None |
| 21 | RHF |
| 22 | FSF |
| 23 | WLA |
| 24 | AVF |
| 25 | VFA |
| 26 | EIR |
| 27 | PW |
| 28 | LEEL |
| 29 | DTR |
| 30 | DTV |
| 31 | TIP |
| 32 | HSP |
| 33 | YPS |
| 34 | LFE |
| 35 | FPS |
| 36 | HPD |
| 37 | TRR |
| 38 | FSH |
| 39 | HTR |
| 40 | RYF |
| 41 | IER |
| 42 | HPS |
| 43 | HHS |
| 44 | HFE |
| 45 | EYR |
| 46 | RRF |
| 47 | DFP |
| 48 | YFS |
| 49 | WEV |
| 50 | FHS |
| 51 | FSI |
| 52 | YFS |
| 53 | FP |
| 54 | WVS |
| 55 | AYI |
| 56 | AWV |
| 57 | ART |
| 58 | ITL |
| 59 | NAF |
| 60 | WLS |
| 61 | HAR |
| 62 | YFS |
| 63 | AFT |
| 64 | ATF |
| 65 | DDS |
| 66 | FFS |
| 67 | LEF |
| 68 | FLR |
| 69 | IQS |
| 70 | IYA |
| 71 | LLP |
| 72 | LPI |
| 73 | YFS |
| 74 | WAL |
| 75 | WRE |
| 76 | WSV |
| 77 | YAI |
| 78 | AYI |
| 79 | ALL |
| 80 | LLL |
| 81 | LLV |
| 82 | LQL |
| 83 | PHY |
| 84 | RIF |
| 85 | VWR |
| 86 | ILL |
| 87 | LAY |
| 88 | AFS |
| 89 | AHP |
| 90 | TSF |
| 91 | VRL |
| 92 | QFA |
| 93 | LEF |
| 94 | VFA |

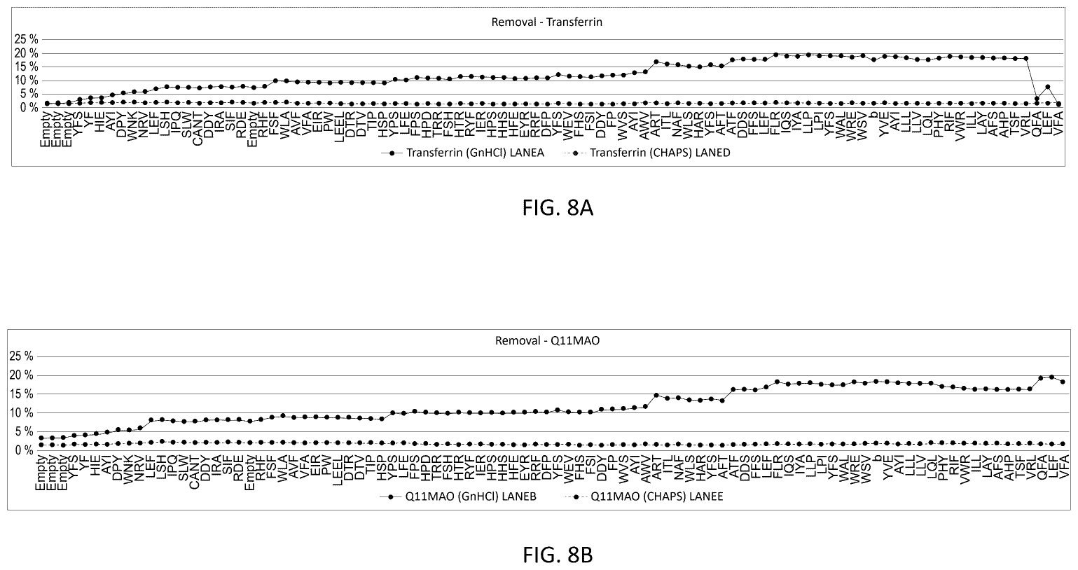

The percentage of dissociation for each cycle was calculated as the difference between the quantity of observed fluorescent signals detected before incubation with the dissociation medium, minus the quantity of observed fluorescent signals detected after incubation with the dissociation medium divided by the total quantity of observed fluorescent signals detected before incubation with the dissociation medium. Two dissociation mediums were tested, one for each of the protein molecule arrays: 1) 100 mM CHAPS in water; and 2) 5.2M guanidinium hydrochloride, 20 mM sodium acetate, and 20 mM magnesium chloride. FIGS. 8A and 8B plot observed dissociation data for the array containing transferrin molecules and the array containing Q11MAO, respectively. Light grey data points are for the CHAPS dissociation medium and dark grey data points are for the guanidinium hydrochloride dissociation medium.

For arrays incubated with the CHAPS dissociation medium, both arrays are observed to have an increasing quantity of dissociation failure due to the accumulation of non-dissociated binding reagents over the 90+ cycles. However, certain binding reagents can be observed to produce larger step changes in removal failure for the transferrin molecules relative to Q11MAO, such as the LEF- and ATF-specific binding reagents. Alternatively, arrays incubated with the GnHCl dissociation medium have a very steady level of dissociation failure across all cycles. Accordingly, dissociation of LEF-specific binding reagents or ATF-specific binding reagents in a CHAPS medium may provide data that facilitates discrimination of transferrin molecules from Q11MAO molecules in a sample. Subsequently, GnHCl can be utilized to successively dissociate binding reagents after CHAPS incubation.

1. A method, comprising:
(a) detecting in a first fluidic medium the presence or absence of binding of binding reagents of a first plurality of binding reagents to a plurality of molecules, wherein the first fluidic medium has a first fluidic condition;
(b) detecting in a second fluidic medium the presence or absence of binding of the binding reagents of a second plurality of binding reagents to the plurality of molecules, wherein the second fluidic medium has a second fluidic condition, wherein the first fluidic condition of the first fluidic medium differs from the second fluidic condition of the second fluidic medium; and
(c) characterizing an individual molecule of the plurality of molecules based upon a binding pattern of binding reagents of the plurality of binding reagents to the individual molecule of the plurality of molecules, wherein the binding pattern comprises a presence or absence of binding of a binding reagent of the plurality of binding reagents to the individual molecule for the first fluidic condition and the second fluidic condition.

2. The method of claim 1, further comprising delivering from a first vessel the first fluidic medium and the first plurality of binding reagents to the plurality of molecules.

3. The method of claim 2, further comprising delivering to the first fluidic medium a fluid component, thereby forming the second fluidic medium having the second fluidic condition.

4. The method of claim 3, wherein the fluid component comprises a diluent, a solvent, a co-solvent, an ionic compound, a buffering species, a surfactant species, a chaotropic species, an acidifying agent, an alkalizing agent, a blocking agent, a kosmotropic species, an excipient reagent, additional binding reagents, or a combination thereof.

5. The method of claim 2, further comprising altering a physical property of the first fluidic medium, thereby forming the second fluidic medium having the second fluidic condition.

6. The method of claim 5, wherein altering the first fluidic medium comprises altering in the first fluidic medium the fluid temperature, fluid pressure, fluid velocity, fluid depth or film thickness, fluid magnetic field magnitude or orientation, fluid electrical field magnitude or orientation, or a combination thereof.

7. The method of claim 2, further comprising: (i) removing the first fluidic medium and the first plurality of binding reagents from the plurality of molecules; and (ii) delivering from a second vessel the second fluidic medium and the second plurality of binding reagents to the plurality of molecules.

8. The method of claim 7, wherein the first plurality of binding reagents is identical to the second plurality of binding reagents.

9. The method of claim 7, wherein the first plurality of binding reagents differs from the second plurality of binding reagents with respect to binding specificity or binding reagent structure.

10. The method of claim 1, further comprising: (i) removing from a first vessel a first volume of fluid comprising the first plurality of binding reagents; (ii) delivering the first volume of fluid to the plurality of molecules, thereby providing the first fluidic medium; (iii) removing from the first vessel a second volume of fluid comprising the second plurality of binding reagents; and (iv) delivering the second volume of fluid to the plurality of molecules, thereby providing the second fluidic medium.

11. The method of claim 10, further comprising, before delivering the second volume of fluid to the plurality of molecules, adding a chemical component to the second volume of fluid.

12. The method of claim 11, wherein the chemical component comprises a diluent, a solvent, a co-solvent, an ionic compound, a buffering species, a surfactant species, a chaotropic species, an acidifying agent, an alkalizing agent, a blocking agent, a kosmotropic species, an excipient reagent, additional binding reagents, or a combination thereof.

13. The method of claim 10, further comprising, before delivering the second volume of fluid to the plurality of molecules, altering a physical property of the second volume of fluid.

14. The method of claim 13, wherein altering the physical property of the second volume of fluid comprises altering in the second volume of fluid the fluid temperature, fluid pressure, fluid velocity, fluid depth or film thickness, or a combination thereof.

15. The method of claim 1, wherein detecting in the first fluidic medium or the second fluidic medium the presence or absence of binding of the binding reagents occurs in the presence of unbound binding reagents.

16. (canceled)

17. The method of claim 1, further comprising: (d) detecting in a third fluidic medium presence or absence of binding of binding reagents of a third plurality of binding reagents to the plurality of molecules, wherein the third fluidic medium has a third fluidic condition; and (e) detecting in a fourth fluidic medium presence or absence of binding of the binding reagents of a fourth plurality of binding reagents to the plurality of molecules, wherein the fourth fluidic medium has a fourth fluidic condition, wherein the third fluidic condition differs from the fourth fluidic condition.

18. The method of claim 17, wherein the third fluidic condition or the fourth fluidic condition is the same as the first fluidic condition or the second fluidic condition.

19. The method of claim 17, wherein the binding pattern comprises for the individual molecule of the plurality of molecules a presence or absence of binding of a binding reagent of the plurality of binding reagents to the individual molecule for the first fluidic condition, the second fluidic condition, the third fluidic condition, and the fourth fluidic condition.

20. (canceled)

21. A method, comprising:
(a) detecting in a first fluidic medium the presence or absence of binding of binding reagents of a first plurality of binding reagents to a plurality of molecules, wherein the first fluidic medium has a first fluidic condition;
(b) detecting in a second fluidic medium the presence or absence of binding of the binding reagents of a second plurality of binding reagents to the plurality of molecules, wherein the second fluidic medium has a second fluidic condition, wherein the first fluidic condition differs from the second fluidic condition; and
(c) distinguishing a first molecule of the plurality of molecules from a second molecule of the plurality of molecules based upon a binding pattern of the binding reagents of the plurality of binding reagents to the plurality of molecules, wherein the binding pattern comprises for each individual molecule of the plurality of molecules a presence or absence of binding of a binding reagent of the plurality of binding reagents to the individual molecule for the first fluidic condition and the second fluidic condition.

22-23. (canceled)

24. A system for analyte characterization, comprising:
(a) a fluidic vessel comprising a plurality of molecules;
(b) a binding reagent kit comprising a plurality of vessels, wherein each vessel of the plurality of vessels comprises a fluidic medium comprising a plurality of binding reagents;
(c) a fluidic system that is configured to transfer the fluidic medium comprising the plurality of binding reagents to the fluidic vessel comprising the plurality of molecules; and
(d) a detection system that is configured to detect at single-analyte resolution for each individual molecule of the plurality of molecules a presence or absence of binding of a binding reagent of the plurality of binding reagents.

25-36. (canceled)
